(19) United States
(12) Patent Application Publication
(10) Pub. No.: US 2008/0299188 A1
(43) Pub. Date: Dec. 4, 2008

(54) CONTROLLED RELEASE DOSAGE FORMS COMBINING IMMEDIATE RELEASE AND SUSTAINTED RELEASE OF LOW-SOLUBILITY DRUG

(75) Inventors: Leah E. Appel, Bend, OR (US); Dwayne T. Friesen, Bend, OR (US); Scott M. Herbig, East Lyme, CT (US); Avinash G. Thombre, East Lyme, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc.

(21) Appl. No.: 11/748,134

(22) Filed: May 14, 2007

(51) Int. Cl. A61K 9/52; A61K 9/00; A61K 31/496; A61P 25/00; A61K 9/14

(52) U.S. Cl. 424/457; 424/400; 424/489; 424/484; 424/490; 514/254.04

(57) ABSTRACT

A controlled release dosage form comprises an immediate release portion and an enteric coated sustained release core.

### BACKGROUND

The present invention relates to a controlled release dosage form having an immediate release portion and an enteric coated sustained release core.

It is well known that for some drugs, controlled release of the drug over time may offer a number of advantages relative to immediate release of the drug. The primary goal of a controlled release dosage form is to maintain the desired therapeutic effect for an extended period of time. Accordingly, such dosage forms should result in a drug concentration in the blood that is greater than the effective or therapeutic concentration for longer periods of time than a corresponding immediate release dosage form containing the same amount of drug. The controlled release dosage forms often result in the reduction or elimination of fluctuations in drug concentration in the blood, which improves disease state management. In addition, because the controlled release dosage form reduces the maximum concentration of drug in the blood relative to an immediate release formulation of the same dose, the controlled release formulation may minimize side effects, and may result in less potentiation or reduction in drug activity with chronic use. Finally, the controlled release dosage form may enhance patient compliance, due to a reduction in dosing frequency, reduction in side effects, or both.

However, for some low-solubility drugs, it is difficult to formulate the drug into a controlled release oral dosage form that sustains the concentration of drug in the blood above the effective concentration for long periods of time. In particular, this problem exists for low-solubility drugs that have a relatively short biological half-life and which are poorly absorbed in either some or all of the lower gastrointestinal tract (e.g., the distal small intestine and colon). For these drugs, the combination of low-solubility, short absorption window, and relatively fast clearance from the blood all work against achieving high blood concentration of the drug for long periods of time. The low-solubility of the drug limits absorption due to the low concentration of the drug in the aqueous environment of the lower gastrointestinal tract. Because the drug is poorly absorbed over some or all of the lower gastrointestinal tract, the period during which the drug may be absorbed may be relatively short. For drugs that are poorly absorbed over the length of the lower gastrointestinal tract (lower small intestine and colon), the period of good absorption may be limited to the upper portion of the small intestine. In such cases, a drug delivered from a controlled release dosage form may cease to be well absorbed soon (1 to 2 hours) after exiting from the stomach. Finally, the relatively short biological half-life means that even if relatively high drug concentration in the blood is achieved initially, the drug concentration in the blood will decline rapidly over time unless a means is found to provide continued absorption of the drug at a rate that is fast enough to overcome the clearance rate at a therapeutic blood level.

Accordingly, there is a continuing need for a controlled release oral dosage form that provides effective concentration of drug in the blood for relatively long periods of time for low-solubility drugs that are poorly absorbed over at least a portion of the gastrointestinal tract and that have a relatively short biological half life.

### SUMMARY OF THE INVENTION

In one aspect, a controlled release oral dosage form comprises an immediate release portion comprising a low-solubility drug and a sustained release core comprising the low-solubility drug. The low-solubility drug has a dose to aqueous solubility ratio of at least about 100 ml. The sustained release core is surrounded by an enteric coating. The sustained release core is sufficiently large so as to be retained in the stomach and provide a delayed release of the drug. The sustained release core alone releases at least 90 wt % of the drug in the core over a release period of from about 1 hour to about 8 hours, and the drug in the sustained release core is in a solubility-improved form.

In another aspect of the invention, a controlled release oral dosage form comprises an immediate release portion comprising a low-solubility drug and a sustained release core comprising the low-solubility drug. The low-solubility drug has a dose to aqueous solubility ratio of at least about 10 ml. The sustained release core is surrounded by an enteric coating. The sustained release core is sufficiently large, so as to be retained in the stomach and provide a delayed release of the drug. The sustained release core releases at least 90 wt % of the drug in the core over a release period of from about 1 hour to about 8 hours, and the drug has a clearance half life of less than about 12 hours.

In one embodiment, the sustained release core and coating collectively have a mass of at least 400 mg.

In another embodiment, the sustained core and enteric coating collectively have a largest dimension of at least 5 mm.

The inventors solved the problem of providing effective concentration of drug in the blood for long periods of time for low-solubility drugs that are poorly absorbed in some or all of the lower gastrointestinal tract and have a relatively short biological half-life as follows. First, the dosage form should have an immediate release portion to immediately release drug to the stomach. This provides an initial burst of drug that results in an initial period of good absorption of the drug and resulting elevated concentration of drug in the blood. Second, the dosage form should be retained in the stomach for as long as possible. This may be accomplished by having a relatively large core (e.g., at least about 400 mg) that does not substantially erode while in the stomach. The sustained release core is also surrounded by an enteric coating that prevents the sustained release core from dissolving or eroding in the stomach. Optionally, it is also generally preferred that the drug be administered in the fed state to maximize retention in the stomach. Although drug present in the stomach is not generally absorbed well from the stomach, drug released from the dosage form while the dosage form is in the stomach serves to deliver drug to the upper small intestines over a prolonged period of time leading to a prolonged period of good absorption of drug.

Third, the drug is released in the lower gastrointestinal tract (lower small intestine and optionally the colon) in a form and over a period of time that allows good absorption. It is necessary that the drug have at least some absorption in the lower gastrointestinal tract. For drugs that are poorly absorbed in the distal small intestine, it is necessary to formulate the drug in the sustained release core into a solubility-improved form so that the absorption of drug in the lower gastrointestinal tract is acceptable. These drugs tend to have a dose-to-solubility ratio of greater than about 100 ml. It is also desired that the sustained release core release the drug over a release period that corresponds to the period of time during which the dosage form is releasing drug (which may be in a solubility-improved form) in a region of the GI tract in which the drug has good absorption. In general, the release period is about 1-8 hours following exit from the stomach for drugs that may have some absorption in both the distal small intestine and the colon (due to the drug being in a solubility improved form), to about 1-4 hours for drugs that have good absorption in the small intestine but are poorly absorbed in the colon.

In one embodiment, the dosage form comprises an enteric coated sustained release core in the form of a matrix device. The immediate release portion is in the form of an immediate release coating.

In another embodiment, the dosage form comprises an enteric coated sustained release core in the form of an osmotic controlled-release device. The immediate release portion is in the form of an immediate release coating.

In yet another embodiment, the dosage form comprises a capsule, the capsule comprising an enteric coated sustained release core and an immediate release portion.

The foregoing and other objectives, features, and advantages of the invention will be more readily understood upon consideration of the following detailed description of the invention.

### BRIEF DESCRIPTION OF THE DRAWINGS

FIG. 1 is a schematic cross section of an exemplary dosage form of the present invention.

FIG. 2 is a schematic cross section of another exemplary dosage form of the present invention.

FIG. 3 is a schematic cross section of yet another exemplary dosage form of the present invention.

FIG. 4 is a schematic cross section of yet another exemplary dosage form of the present invention.

### DETAILED DESCRIPTION OF THE PREFERRED EMBODIMENTS

The controlled release dosage forms of the present invention increase the length of time during which the concentration of drug in the blood (serum or plasma) is greater than the effective concentration. The controlled release dosage forms achieve this by providing both a sustained release core and an immediate release portion. The sustained release core is surrounded by an enteric coating. The nature of the drugs for which the dosage forms are suitable, the sustained release cores, the enteric coatings, and the immediate release portion are described in more detail below.

### The Drug

The term “drug” is conventional, denoting a compound having beneficial prophylactic and/or therapeutic properties when administered to an animal, especially humans. The drug has a relatively fast clearance rate. By “clearance half life” is meant the time required for the body to clear the drug from the blood, so that the concentration of drug in the blood (serum or plasma) decreases by one-half. The clearance half life may be determined, for example, by measuring concentration of drug in the blood after administration of the drug via intravenous infusion and fitting the data using a first order single compartment pharmacokinetic model. See, e.g., Pharmacokinetics and Metabolism in Drug Design, Smith et al., (Wiley-VCH 2001) at pages 20-21. The clearance half life is less than about 12 hours. In general, the invention has increasing utility as the clearance half life decreases. The clearance half life may be less than about 8 hours, less than about 6 hours, or even less than about 4 hours. Nevertheless, the clearance half life should be great enough (e.g., greater than about 1 hour) so that the drug may be absorbed and accumulate in the blood without being immediately cleared from the body.

By “low-solubility drug” is meant that the drug has a dose to aqueous solubility ratio of greater than about 10 ml, where the drug solubility (mg/mL) is the minimum value observed in any physiologically relevant aqueous solution (e.g., those with pH values between 1 and 8) including USP simulated gastric and intestinal buffers, and dose is in mg. Thus, a dose-to-aqueous solubility ratio may be calculated by dividing the dose (in mg) by the solubility (in mg/mL). The invention has greater utility as the dose to aqueous solubility ratio increases. Thus, the dose to aqueous solubility may be greater than about 50 ml, greater than about 100 ml, even greater than about 500 ml, or even greater than about 1000 ml. By “dose” is meant the amount of drug present in the dosage form. By aqueous solubility is meant the minimum aqueous solubility at physiologically relevant pH (e.g., pH 1-8). In general, the minimum aqueous solubility is generally less than 10 mg/mL. The invention has increasing utility as the minimum solubility decreases. The minimum aqueous solubility may be less than 1 mg/mL, less than 0.5 mg/mL, even less than 0.1 mg/mL, or even less than 0.01 mg/mL.

Preferred classes of drugs include, but are not limited to, antihypertensives, antianxiety agents, anticlotting agents, anticonvulsants, blood glucose-lowering agents, decongestants, antihistamines, antitussives, antineoplastics, beta blockers, anti-inflammatories, antipsychotic agents, cognitive enhancers, cholesterol-reducing agents, anti-atherosclerotic agents, antiobesity agents, autoimmune disorder agents, anti-impotence agents, antibacterial and antifungal agents, hypnotic agents, anti-Parkinsonism agents, anti-Alzheimer's disease agents, antibiotics, anti-depressants, antiviral agents, glycogen phosphorylase inhibitors, and cholesteryl ester transfer protein inhibitors.

The drug may be in any pharmaceutically acceptable form. By “pharmaceutically acceptable form” is meant any pharmaceutically acceptable derivative or variation, including stereoisomers, stereoisomer mixtures, enantiomers, solvates, hydrates, isomorphs, polymorphs, pseudomorphs, neutral forms, salt forms and prodrugs.

One class of drugs that may benefit from the invention are drugs which are relatively well absorbed in the upper small intestine, but poorly absorbed in the distal small intestine or colon. Such drugs generally have a dose to solubility ratio in the range of from about 10 ml to 100 ml or more. Such drugs are often polar (i.e., characterized by a dipole). Such drugs may have a log P of less than about 3.0, or even less than about 2.0. Exemplary classes of such drugs include antivirals and antibiotics.

Another class of drugs that may benefit from the invention are those drugs that have low-solubility and are poorly absorbed in the small intestine and colon, but which are capable of being formulated so as to be better absorbed in the small intestine. Such drugs generally have a dose to solubility ratio greater than about 100 ml. For this class of drugs, the drug is formulated in a solubility improved form so as to improve absorption of the drug in the small intestine. The term “solubility-improved form” refers to a form of the drug alone that, when delivered to an in vivo environment of use (such as, for example, the gastrointestinal tract of a mammal) or a physiologically relevant in vitro solution (such as phosphate buffered saline or a Model Fasted Duodenal solution described below) provides concentration enhancement of the drug as described in more detail below. Often, solubility-improved forms dissolve to a concentration in excess of their equilibrium solubility but then precipitate or crystallize such that their dissolved concentration approaches the equilibrium concentration. Examples of “solubility-improved forms” include but are not limited to: (1) a crystalline highly soluble form of the drug such as a salt; (2) a high-energy crystalline form of the drug; (3) a hydrate or solvate crystalline form of a drug; (4) an amorphous form of a drug (for a drug that may exist as either amorphous or crystalline); (5) drug particles having reduced or small particle size; (6) nanoparticles of drug; (7) combination of the drug with a cyclodextrin; (8) combination of the drug and a solubilizing agent; (9) amorphous forms of the drug such as solid amorphous dispersions or adsorbates of amorphous drug; and (10) semi-ordered forms of the drug.

In one aspect of the invention, the solubility-improved form of the drug is crystalline and is a highly soluble salt form of the drug. As used herein, “highly soluble salt form” means that the drug is in a salt form that provides in at least one in vitro test medium a maximum concentration of the drug that is greater than the equilibrium concentration provided by the lowest solubility form of the drug. The drug can be any pharmaceutically acceptable salt form of a basic, acidic, or zwitterionic drug that meets this criteria. Examples of salt forms for basic drugs include the chloride, bromide, acetate, iodide, mesylate, phosphate, maleate, citrate, sulfate, tartrate, lactate salts and the like. Examples of salt forms for acidic drugs include the sodium, calcium, potassium, zinc, magnesium, lithium, aluminum, meglumine, diethanolamine, benzathine, choline, and procaine salts and the like. These salts can also be used for zwitterionic drugs.

An example of a drug having a crystalline highly soluble salt form is ziprasidone. Ziprasidone hydrochloride monohydrate has a solubility of about 10 μgA/mL (expressed as the free base) in phosphate buffered saline (pH 6.5), whereas the free base form has a solubility of less than about 0.2 μgA/mL under the same conditions. Thus, crystalline ziprasidone hydrochloride is a solubility-improved form relative to the crystalline free base form of drug.

Alternatively, in another separate aspect of the invention, the drug exists in a high-energy crystalline form that has improved solubility relative to a low-energy crystalline form. It is known that some drugs may crystallize into one of several different crystal forms. Such crystal forms are often referred to as “polymorphs.” As used herein, “a high-energy crystalline form” means that the drug is in a crystal form which provides concentration enhancement as described below. Such high-energy crystalline forms often dissolve and then precipitate or crystallize from solution in a lower energy state. The concentration of dissolved drug ultimately approaches its equilibrium concentration.

In yet another separate aspect of the invention, although the drug may be capable of existing in either the amorphous or crystalline form, in the composition the solubility-improved form is the amorphous form. Preferably, at least a major portion of the drug is amorphous. By “amorphous” is meant simply that the drug is in a non-crystalline state. As used herein, the term “a major portion” of means that at least 60 wt % of the drug in the composition is in the amorphous form, rather than the crystalline form. Preferably, the drug is substantially amorphous. As used herein, “substantially amorphous” means that the amount of drug in crystalline form does not exceed about 25 wt %. More preferably, the drug is “almost completely amorphous,” meaning that the amount of drug in the crystalline form does not exceed about 10 wt %. Amounts of crystalline drug may be measured by Powder X-Ray Diffraction (PXRD), Scanning Electron Microscope (SEM) analysis, differential scanning calorimetry (DSC), or any other standard quantitative measurement.

The amorphous form of the drug may be any form in which the drug is amorphous. Examples of amorphous forms of drug include solid amorphous dispersions of drug in a polymer, such as disclosed in commonly assigned US published patent application 2002/0009494A1 herein incorporated by reference. Alternatively, the drug may be adsorbed in amorphous form on a solid substrate, such as disclosed in commonly assigned US published patent application 2003/0054037A1, herein incorporated by reference. As yet another alternative, amorphous drug may be stabilized using a matrix material, such as disclosed in commonly assigned US Patent application 2003/0104063A1, herein incorporated by reference.

In yet another embodiment, the solubility-improved form comprises drug particles that are sufficiently small that they improve the dissolution rate of the drug relative to the bulk (large) crystalline form of the drug. By small particle size is meant that the drug particles have a mean diameter of less than 50 microns, more preferably less than 20 microns, and even more preferably less than 10 microns. A particularly preferred and simple method for forming small particles of drug involves breaking larger diameter particles into smaller diameter particles. Particle size reduction may be accomplished by any conventional method, such as by milling, or grinding. Exemplary milling devices include a chaser mill, ball mill, vibrating ball mill, hammer mill, impact grinding mill, fluid energy mill (jet mill), and centrifugal-impact pulverizers. Alternatively, small particles may be formed by atomization or precipitation. One method for reducing the drug particle size is jet milling. Small drug particles can also be formed by other means, such as dissolution in a solvent such as alcohol or water followed by precipitation by mixing with a non-solvent. Another method to reduce particle size is by melting or dissolving the drug in a solvent and atomizing the resulting liquid by spray congealing or spray drying to form a powder. Other methods for making small crystalline drug particles include wet milling or processing in a homogenizer. The size of the drug particles needed to enhance drug dissolution compared with the bulk crystalline form of the drug will depend on the particular drug. In general, however, dissolution rate tends to increase as the drug particle size decreases. For example, in the case of the drug ziprasidone, jet-milled drug may have a mean particle size of less than about 10 microns, and more preferably less than about 5 microns.

In another embodiment, the drug may be in the form of nanoparticles. The term “nanoparticle” refers to drug in the form of particles generally having an effective average particle size of less than about 500 nm, more preferably less than about 250 nm and even more preferably less than about 100 nm. Examples of such nanoparticles are further described in U.S. Pat. No. 5,145,684.

The nanoparticles of the drug can be prepared using any known method for preparing nanoparticles. One method comprises suspending drug in a liquid dispersion medium and applying mechanical means in the presence of grinding media to reduce the particle size of the drug substance to the effective average particle size. The particles can be reduced in size in the presence of a surface modifier. Alternatively, the particles can be contacted with a surface modifier after attrition. Other alternative methods for forming nanoparticles are described in U.S. Pat. No. 5,560,932, and U.S. Pat. No. 5,874,029, both incorporated-herein by reference.

Another solubility-improved form of drug comprises drug in combination with a cyclodextrin. As used herein, the term “cyclodextrin” refers to all forms and derivatives of cyclodextrin. Particular examples of cyclodextrin include α-cyclodextrin, β-cyclodextrin, and γ-cyclodextrin. Exemplary derivatives of cyclodextrin include mono- or polyalkylated β-cyclodextrin, mono- or polyhydroxyalkylated β-cyclodextrin, hydroxypropyl β-cyclodextrin (hydroxypropylcyclodextrin), mono, tetra or hepta-substituted β-cyclodextrin, sulfoalkyl ether cyclodextrin (SAE-CD), and sulfobutylether cyclodextrin (SBECD).

These solubility-improved forms, also known as cyclodextrin derivatives, herein after referred to as “cyclodextrin/drug forms” can be simple physical mixtures. An example of such is found in U.S. Pat. No. 5,134,127, herein incorporated by reference. Alternatively, the drug and cyclodextrin may be complexed together. For example, the active drug and sulfoalkyl ether cyclodextrin (SAE-CD) may be preformed into a complex prior to the preparation of the final formulation. Alternatively, the drug can be formulated by using a film coating surrounding a solid core comprising a release rate modifier and a SAE-CD/drug mixture, as disclosed in U.S. Pat. No. 6,046,177, herein incorporated by reference. Upon exposure in the use environment, the SAE-CD/drug mixture converts at least partially to a complex. Alternatively, sustained-release formulations containing SAE-CD may consist of a core comprising a physical mixture of one or more SAE-CD derivative, an optional release rate modifier, a therapeutic agent, a major portion of which is not complexed to the SAE-CD, and an optional release rate modifying coating surrounding the core. Other cyclodextrin/drug forms contemplated by the invention are found in U.S. Pat. Nos. 5,134,127, 5,874,418, and 5,376,645, all of which are incorporated by reference.

Another solubility-improved form of drug is a combination of drug and a solubilizing agent. Such solubilizing agents promote the aqueous solubility of drug. When drug is administered to an aqueous use environment in the presence of the solubilizing agent, the concentration of dissolved drug may exceed the equilibrium concentration of dissolved drug, at least temporarily. Examples of solubilizing agents include surfactants; pH control agents such as buffers, organic acids, and organic acid salts; glycerides; partial glycerides; glyceride derivatives; polyoxyethylene and polyoxypropylene ethers and their copolymers; sorbitan esters; polyoxyethylene sorbitan esters; carbonate salts; alkyl sulfonates; and phospholipids. In this aspect, the drug and solubilizing agent are both preferably solid.

Exemplary surfactants include fatty acid and alkyl sulfonates; commercial surfactants such as benzalkonium chloride (HYAMINE® 1622, available from Lonza, Inc., Fairlawn, N.J.); dioctyl sodium sulfosuccinate (DOCUSATE SODIUM, available from Mallinckrodt Spec. Chem., St. Louis, Mo.); polyoxyethylene sorbitan fatty acid esters (TWEEN®, available from ICI Americas Inc., Wilmington, Del.; LIPOSORB® O-20, available from Lipochem Inc., Patterson N.J.; CAPMUL® POE-0, available from Abitec Corp., Janesville, Wis.); and natural surfactants such as sodium taurocholic acid, 1-palmitoyl-2-oleoyl-sn-glycero-3-phosphocholine, lecithin, and other phospholipids and mono- and diglycerides.

Another class of solubilizing agents consists of organic acids and organic acid salts. Exemplary organic acids include acetic, aconitic, adipic, ascorbic, aspartic, benzenesulfonic, benzoic, camphorsulfonic, cholic, citric, decanoic, erythorbic, 1,2-ethanedisulfonic, ethanesulfonic, formic, fumaric, gluconic, glucuronic, glutamic, glutaric, glyoxylic, heptanoic, hippuric, hydroxyethanesulfonic, lactic, lactobionic, levulinic, lysine, maleic, malic, malonic, mandelic, methanesulfonic, mucic, 1- and 2-naphthalenesulfonic, nicotinic, pamoic, pantothenic, phenylalanine, 3-phenylpropionic, phthalic, salicylic, saccharic, succinic, tannic, tartaric, p-toluenesulfonic, tryptophan, and uric.

Yet another class of solubilizing agents consists of lipophilic microphase-forming materials described in US published patent application 2003/0228358A1 published Dec. 11, 2003 herein incorporated by reference. Lipophilic microphase-forming material may comprise a surfactant and/or a lipophilic material. Thus, as used herein, the “lipophilic microphase-forming material” is intended to include blends of materials in addition to a single material. Examples of amphiphilic materials suitable for use as the lipophilic microphase-forming material include: sulfonated hydrocarbons and their salts, such as sodium 1,4-bis(2-ethylhexyl) sulfosuccinate, also known as docusate sodium (CROPOL) and sodium lauryl sulfate (SLS); poloxamers, also referred to as polyoxyethylene-polyoxypropylene block copolymers (PLURONICs, LUTROLs); polyoxyethylene alkyl ethers (CREMOPHOR A, BRIJ); polyoxyethylene sorbitan fatty acid esters (polysorbates, TWEEN); short-chain glyceryl mono-alkylates (HODAG, IMWITTOR, MYRJ); polyglycolized glycerides (GELUCIREs); mono- and di-alkylate esters of polyols, such as glycerol; nonionic surfactants such as polyoxyethylene 20 sorbitan monooleate, (polysorbate 80, sold under the trademark TWEEN 80, available commercially from ICI); polyoxyethylene 20 sorbitan monolaurate (Polysorbate 20, TWEEN 20); polyethylene (40 or 60) hydrogenated castor oil (available under the trademarks CREMOPHOR® RH40 and RH60 from BASF); polyoxyethylene (35) castor oil (CREMOPHOR® EL); polyethylene (60) hydrogenated castor oil (Nikkol HCO-60); alpha tocopheryl polyethylene glycol 1000 succinate (Vitamin E TPGS); glyceryl PEG 8 caprylate/caprate (available commercially under the registered trademark LABRASOL® from Gattefosse); PEG 32 glyceryl laurate (sold commercially under the registered trademark GELUCIRE 44/14 by Gattefosse), polyoxyethylene fatty acid esters (available commercially under the registered trademark MYRJ from ICI), polyoxyethylene fatty acid ethers (available commercially under the registered trademark BRIJ from ICI). Alkylate esters of polyols may be considered amphiphilic or hydrophobic depending on the number of alkylates per molecule and the number of carbons in the alkylate. When the polyol is glycerol, mono- and di-alkylates are often considered amphiphilic while trialkylates of glycerol are generally considered hydrophobic. However, some scientists classify even medium chain mono- and di-glycerides as hydrophobic. See for example Patel et al U.S. Pat. No. 6,294,192 (B1), which is incorporated herein in its entirety by reference. Regardless of the classification, compositions comprising mono- and di-glycerides are preferred compositions of this invention. Other suitable amphiphilic materials may be found in Patel, U.S. Pat. No. 6,294,192 and are listed as “hydrophobic non-ionic surfactants and hydrophilic ionic surfactants.”

It should be noted that some amphiphilic materials may not be water immiscible by themselves, but instead are at least somewhat water soluble. Such amphiphilic materials may nevertheless be used in mixtures to form the lipophilic microphase, particularly when used as mixtures with hydrophobic materials.

Examples of hydrophobic materials suitable for use as the lipophilic microphase-forming material include: medium-chain glyceryl mono-, di-, and tri-alkylates (CAPMUL MCM, MIGLYOL 810, MYVEROL 18-92, ARLACEL 186, fractionated coconut oil, light vegetable oils); sorbitan esters (ARLACEL 20, ARLACEL 40); long-chain fatty alcohols (stearyl alcohol, cetyl alcohol, cetostearyl alcohol); long-chain fatty-acids (stearic acid); and phospholipids (egg lecithin, soybean lecithin, vegetable lecithin, sodium taurocholic acid, and 1,2-diacyl-sn-glycero-3-phosphocholine, such as 1-palmitoyl-2-oleyl-sn-glycero-3-phosphocoline, 1,2-dipalmitoyl-sn-glycero-3-phosphocholine, 1,2-distearoyl-sn-glycero-3-phosphocholine, 1-plamitoyl-2-stearoyl-sn-glycero-3-phosphocholine, and other natural or synthetic phosphatidyl cholines); mono and diglycerides of capric and caprylic acid under the following registered trademarks: Capmul® MCM, MCM 8, and MCM 10, available commercially from Abitec, and Imwitor® 988, 742 or 308, available commercially from Condea Vista; polyoxyethylene 6 apricot kernel oil, available under the registered trademark Labrafil® M 1944 CS from Gattefosse; polyoxyethylene corn oil, available commercially as Labrafil® M 2125; propylene glycol monolaurate, available commercially as Lauroglycol from Gattefosse; propylene glycol dicaprylate/caprate available commercially as Captex® 200 from Abitec or Miglyol® 840 from Condea Vista, polyglyceryl oleate available commercially as Plurol oleique from Gattefosse, sorbitan esters of fatty acids (e.g., Span® 20, Crill® 1, Crill® 4, available commercially from ICI and Croda), and glyceryl monooleate (Maisine, Peceol); medium chain triglycerides (MCT, C6-C12) and long chain triglycerides (LCT, C14-C20) and mixtures of mono-, di-, and triglycerides, or lipophilic derivatives of fatty acids such as esters with alkyl alcohols; fractionated coconut oils, such as Miglyol® 812 which is a 56% caprylic (C8) and 36% capric (C10) triglyceride, Miglyol® 810 (68% C8 and 28% C10), Neobee® M5, Captex® 300, Captex® 355, and Crodamol® GTCC; (Miglyols are supplied by Condea Vista Inc. (Huls), Neobee® by Stepan Europe, Voreppe, France, Captex by Abitec Corp., and Crodamol by Croda Corp); vegetable oils such as soybean, safflower, corn, olive, cottonseed, arachis, sunflowerseed, palm, or rapeseed; fatty acid esters of alkyl alcohols such as ethyl oleate and glyceryl monooleate. Other hydrophobic materials suitable for use as the lipophilic microphase-forming material include those listed in Patel, U.S. Pat. No. 6,294,192 as “hydrophobic surfactants.” Exemplary classes of hydrophobic materials include: fatty alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; and mixtures thereof. Mixtures of relatively hydrophilic materials, such as those termed herein as “amphiphilic” or in Patel as “hydrophilic surfactants” and the above hydrophobic materials are particularly suitable. Specifically, the mixtures of hydrophobic surfactants and hydrophilic surfactants disclosed by Patel are suitable and for many compositions, preferred. However, unlike Patel, mixtures that include triglycerides as a hydrophobic component are also suitable.

In one embodiment, the lipophilic microphase-forming material is selected from the group consisting of polyglycolized glycerides (GELUCIREs); polyethylene (40 or 60) hydrogenated castor oil (available under the trademarks CREMOPHOR® RH40 and RH60 from BASF); polyoxyethylene (35) castor oil (CREMOPHOR® EL); polyethylene (60) hydrogenated castor oil (Nikkol HCO-60); alpha tocopheryl polyethylene glycol 1000 succinate (Vitamin E TPGS); glyceryl PEG 8 caprylate/caprate (available commercially under the registered trademark LABRASOL® from Gattefosse); PEG 32 glyceryl laurate (sold commercially under the registered trademark GELUCIRE 44/14 by Gattefosse); polyoxyethylenefatty acid esters (available commercially under the registered trademark MYRJ from ICI); polyoxyethylene fatty acid ethers (available commercially under the registered trademark BRIJ from ICI); polyoxyethylene-polyoxypropylene block copolymers (PLURONICs, LUTROLs); polyoxyethylene alkyl ethers (CREMOPHOR A, BRIJ); long-chain fatty alcohols (stearyl alcohol, cetyl alcohol, cetostearyl alcohol); long-chain fatty-acids (stearic acid); polyoxyethylene 6 apricot kernel oil, available under the registered trademark Labrafil® M 1944 CS from Gattefosse; polyoxyethylene corn oil, available commercially as Labrafil® M 2125; propylene glycol monolaurate, available commercially as Lauroglycol from Gattefosse; polyglyceryl oleate available commercially as Plurol oleique from Gattefosse; triglycerides, including medium chain triglycerides (MCT, C₆-C₁₂) and long chain triglycerides (LCT, C₁₄-C₂₀); fractionated coconut oils, such as Miglyol® 812 which is a 56% caprylic (C₈) and 36% capric (C₁₀) triglyceride, Miglyol® 810 (68% C₈ and 28% C₁₀), Neobee® M5, Captex® 300, Captex® 355, and Crodamol® GTCC; (Miglyols are supplied by Condea Vista Inc. [Huls], Neobee® by Stepan Europe, Voreppe, France, Captex by Abitec Corp., and Crodamol by Croda Corp); vegetable oils such as soybean, safflower, corn, olive, cottonseed, arachis, sunflowerseed, palm, or rapeseed; polyoxyethylene alkylethers; fatty acids; lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; transesterified vegetable oils; sterols; sterol derivatives; sugar esters; sugar ethers; sucroglycerides; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; and mixtures thereof.

Especially preferred lipophilic microphase-forming materials include mixtures of polyethoxylated castor oils and medium-chain glyceryl mono-, di-, and/or tri-alkylates, (such as mixtures of CREMOPHOR RH40 and CAPMUL MCM), mixtures of polyoxyethylene sorbitan fatty acid esters and medium-chain glyceryl mono-, di-, and/or tri-alkylates, (such as mixtures of TWEEN 80 and CAPMUL MCM), mixtures of polyethoxylated castor oils and medium-chain glyceryl mono-, di-, and/or tri-alkylates, (such as mixtures of CREMOPHOR RH40 and ARLACEL 20), mixtures of sodium taurocholic acid and palmitoyl-2-oleyl-sn-glycero-3-phosphocholine and other natural or synthetic phosphatidylcholines, and mixtures of polyglycolized glycerides and medium-chain glyceryl mono-, di-, and/or tri-alkylates, (such as mixtures of Gelucire 44/14 and CAPMUL MCM).

Another solubility-improved form of drug is drug in a semi-ordered state, such as disclosed in commonly assigned U.S. Provisional Patent Application Ser. No. 60/403,087 filed Aug. 12, 2002, herein incorporated by reference.

Several methods, such as an in vitro dissolution test or a membrane permeation test may be used to determine if a form of the drug is a solubility-improved form and the degree of solubility improvement. An in vitro dissolution test may be performed by adding the solubility-improved form of drug to a dissolution test media, such as model fasted duodenal (MFD) solution, phosphate buffered saline (PBS) solution, a simulated intestinal buffer solution, or distilled water and agitating to promote dissolution. An appropriate PBS solution is an aqueous solution comprising 20 mM Na₂HPO₄, 47 mM KH₂PO₄, 87 mM NaCl, and 0.2 mM KCl, adjusted to pH 6.5 with NaOH. An appropriate MFD solution is the same PBS solution wherein there is also present 7.3 mM sodium taurocholic acid and 1.4 mM of 1-palmitoyl-2-oleyl-sn-glycero-3-phosphocholine. An appropriate simulated intestinal buffer solution is 50 mM NaH₂PO₄ and 2 wt % sodium lauryl sulfate, adjusted to pH 7.5. Distilled water is a preferred dissolution media for some fast precipitating salts. In cases where the solubility-improved form is an ionic salt of the drug it is often observed that when neutral buffer solutions (pH 6 to 8) are used, the solubility-improved form rapidly converts to the lowest energy form of the drug, typically the neutral crystalline form. In such cases it may be preferred to use an unbuffered test medium such as distilled water as the dissolution medium.

In one method for evaluating whether the form is a solubility-improved form, the solubility-improved form of the drug when tested in an in vitro dissolution test meets at least one, and preferably both, of the following conditions. The first condition is that the solubility-improved form provides a higher maximum dissolved drug concentration (MDC) of drug in the in vitro dissolution test relative to a control composition consisting of the lowest solubility crystalline form of the drug. That is, once the solubility-improved form is introduced into a use environment, the solubility-improved form provides a higher aqueous concentration of dissolved drug relative to the control composition. The control composition is the lowest solubility, bulk crystalline form of the drug alone. Preferably, the solubility-improved form provides an MDC of drug in aqueous solution that is at least 1.25-fold that of the control composition, more preferably at least 2-fold, and most preferably at least 3-fold. For example, if the MDC provided by the test composition is 22 μg/ml, and the MDC provided by the control composition is 2 μg/ml, the solubility-improved form provides an MDC that is 11-fold that provided by the control composition.

The second condition is that the solubility-improved form provides a higher dissolution area under the concentration versus time curve (AUC) of dissolved drug in the in vitro dissolution test relative to a control composition consisting of an equivalent amount of drug alone. More specifically, in the in vitro use environment, the solubility-improved form provides an AUC for any 90-minute period from about 0 to about 270 minutes following introduction to the use environment that is at least 1.25-fold that of the control composition described above. Preferably, the AUC provided by the composition is at least 2-fold, more preferably at least 3-fold that of the control composition.

An in vitro test to evaluate enhanced drug concentration in aqueous solution can be conducted by (1) adding with agitation a sufficient quantity of control composition, that is, the lowest solubility crystalline drug alone, to the in vitro test medium, such as distilled water or an MFD, PBS, or simulated intestinal buffer solution, to achieve equilibrium concentration of drug; (2) in a separate test, adding with agitation a sufficient quantity of test composition (e.g., the solubility-improved form) in the same test medium, such that if all drug dissolved, the theoretical concentration of drug would exceed the equilibrium concentration provided by the control composition by a factor of at least 2, and preferably by a factor of at least 10; and (3) comparing the measured MDC and/or aqueous AUC of the test composition in the test medium with the equilibrium concentration, and/or with the aqueous AUC of the control composition. In conducting such a dissolution test, the amount of test composition or control composition used is an amount such that if all of the drug dissolved, the drug concentration would be at least 2-fold, preferably at least 10-fold, and most preferably at least 100-fold that of the equilibrium concentration.

The concentration of dissolved drug is typically measured as a function of time by sampling the test medium and plotting drug concentration in the test medium vs. time so that the MDC can be ascertained. The MDC is taken to be the maximum value of dissolved drug measured over the duration of the test. The aqueous AUC is calculated by integrating the concentration versus time curve over any 90-minute time period between the time of introduction of the composition into the aqueous use environment (when time equals zero) and 270 minutes following introduction to the use environment (when time equals 270 minutes). Typically, when the composition reaches its MDC rapidly, (in less than about 30 minutes), the time interval used to calculate AUC is from time equals zero to time equals 90 minutes. However, if the AUC of a composition over any 90-minute time period described above meets the criterion of this invention, then the drug is considered to be in a solubility-improved form.

To avoid large drug particulates that would give an erroneous determination, the test solution is either filtered or centrifuged. “Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred solubility-improved forms. It should be recognized that this definition of “dissolved drug” encompasses not only monomeric solvated drug molecules but also a wide range of species such as polymer/drug assemblies that have submicron dimensions such as drug aggregates, aggregates of mixtures of polymer and drug, micelles, polymeric micelles, colloidal particles or nanocrystals, polymer/drug complexes, and other such drug-containing species that are present in the filtrate or supernatant in the specified dissolution test.

In another method for evaluation of whether a drug form is a solubility-improved form, the dissolution rate of the solubility improved form is measured and compared to the dissolution rate of the bulk crystalline form of the lowest solubility form of the drug. The dissolution rate may be tested in any appropriate dissolution media, such as PBS solution, MFD solution, simulated intestinal buffer solution, or distilled water. Distilled water is a preferred dissolution media for salt forms that rapidly precipitate. The dissolution rate of the solubility-improved form is greater than the dissolution rate of the lowest solubility form of the drug in its bulk crystalline form. Preferably, the dissolution rate is 1.25-fold that of the lowest solubility form of the drug, more preferably at least 2-fold, and even more preferably at least 3-fold that of the lowest solubility form of the drug.

Alternatively, an in vitro membrane-permeation test may be used to determine if the drug is in a solubility-improved form. In this test, the solubility-improved form is placed in, dissolved in, suspended in, or otherwise delivered to the aqueous solution to form a feed solution. The aqueous solution can be any physiologically relevant solution, such as an MFD or PBS or simulated intestinal buffer solution, as described above. After forming the feed solution, the solution may be agitated to dissolve or disperse the solubility-improved form therein or may be added immediately to a feed solution reservoir. Alternatively, the feed solution may be prepared directly in a feed solution reservoir. Preferably, the feed solution is not filtered or centrifuged after administration of the solubility-improved form prior to performing the membrane-permeation test.

The feed solution is then placed in contact with the feed side of a microporous membrane, the feed side surface of the microporous membrane being hydrophilic. The portion of the pores of the membrane that are not hydrophilic are filled with an organic fluid, such as a mixture of decanol and decane, and the permeate side of the membrane is in fluid communication with a permeate solution comprising the organic fluid. Both the feed solution and the organic fluid remain in contact with the microporous membrane for the duration of the test. The length of the test may range from several minutes to several hours or even days.

The rate of transport of drug from the feed solution to the permeate solution is determined by measuring the concentration of drug in the organic fluid in the permeate solution as a function of time or by measuring the concentration of drug in the feed solution as a function of time, or both. This can be accomplished by methods well known in the art, including by use of ultraviolet/visible (UV/Vis) spectroscopic analysis, high-performance liquid chromatography (HPLC), gas chromatography (GC), nuclear magnetic resonance (NMR), infra red (IR) spectroscopic analysis, polarized light, density, and refractive index. The concentration of drug in the organic fluid can be determined by sampling the organic fluid at discrete time points and analyzing for drug concentration or by continuously analyzing the concentration of drug in the organic fluid. For continuous analysis, UV/Vis probes may be used, as can flow-through cells. In all cases, the concentration of drug in the organic fluid is determined by comparing the results against a set of standards, as well known in the art.

From these data, the maximum flux of drug across the membrane is calculated by multiplying the maximum slope of the concentration of drug in the permeate solution versus time plot by the permeate volume and dividing by the membrane area. This maximum slope is typically determined during the first 10 to 90 minutes of the test, where the concentration of drug in the permeate solution often increases at a nearly constant rate following a short time lag of a few minutes. At longer times, as more of the drug is removed from the feed solution, the slope of the concentration versus time plot decreases. Often, the slope approaches zero as the driving force for transport of drug across the membrane approaches zero; that is, the drug in the two phases approaches equilibrium. The maximum flux is determined either from the linear portion of the concentration versus time plot, or is estimated from a tangent to the concentration versus time plot at time where the slope is at its highest value if the curve is non-linear. Further details of this membrane-permeation test are presented in co-pending U.S. Patent Application Ser. No. 60/557,897, entitled “Method and Device for Evaluation of Pharmaceutical Compositions,” filed Mar. 30, 2004 (attorney Docket No. PC25968), incorporated herein by reference.

A typical in vitro membrane-permeation test to evaluate solubility-improved drug forms can be conducted by (1) administering a sufficient quantity of test composition (that is, the solubility-improved form of the drug) to a feed solution, such that if all of the drug dissolved, the theoretical concentration of drug would exceed the equilibrium concentration of the drug by a factor of at least 2; (2) in a separate test, adding an equivalent amount of control composition (that is, the lowest solubility form of the drug) to an equivalent amount of test medium; and (3) determining whether the measured maximum flux of drug provided by the test composition is at least 1.25-fold that provided by the control composition. A composition is a solubility-improved form if, when dosed to an aqueous use environment, it provides a maximum flux of drug in the above test that is at least about 1.25-fold the maximum flux provided by the control composition. Preferably, the maximum flux provided by the compositions are at least about 1.5-fold, more preferably at least about 2-fold, and even more preferably at least about 3-fold that provided by the control composition.

### Sustained Release Core

The dosage form comprises a sustained release core comprising the low-solubility drug. The core is sufficiently large so that the core will be retained in the stomach so as to delay exit of the dosage form from the stomach, thereby providing a delayed release of the drug. Preferably, the dosage form is large enough to provide at least a one hour delay, and more preferably a two to six hour delay or longer. Gastric retention time ranges from approximately two to eight hours when a large, non-eroding or dissolving dosage form is administered in the fed state. Gastric retention is related to the size and mass of the dosage form. In one embodiment, the dosage form has a longest dimension of at least 5 mm, more preferably at least 7 mm, and even more preferably at least 10 mm. For example, a tablet having a height of 5 mm and a diameter of 10 mm would have a longest dimension of 10 mm. In another embodiment, the enteric coated sustained release core has a mass of at least about 400 mg. That is, collectively the core and enteric coating have a total mass of greater than 400 mg. The enteric coated sustained release core may be larger, so long as the resulting dosage form may be conveniently swallowed. Thus, the enteric coated sustained release core may be at least about 500 mg, or may be even at least about 600 mg. In addition, the dosage form may swell by absorbing water upon ingestion thereby increasing in size, which can further promote retention in the stomach.

The sustained release core releases the drug over an extended period of time that coincides with the period during which the drug is absorbed in the lower gastrointestinal tract. Where the drug is well absorbed in the small intestine but poorly absorbed in the colon, the sustained release core releases greater than 90 wt % of the drug over a release period of about 1 to 6 hours, and more preferably about 1 to 4 hours. For those drugs that are well absorbed in both the small intestine and colon when in a solubility improved form, the release period may be from about 1 to 8 hours. By “release period” is meant the time required for the drug to be released from the core in an intestinal use environment once the enteric coating has dissolved.

An in vitro test may be used to determine whether a dosage form has a release period within the scope of the present invention. In vitro tests are well known in the art. An example is a “residual test,” which is described below for sertraline HCl. The dosage form is placed into a stirred USP type 2 dissoette flask, containing 900 mL of a buffer solution simulating the contents of the small intestine (6 mM KH₂PO₄, 64 mM KCl, 35 mM NaCl, pH 7.2, 210 mOsm/kg). The dosage form is placed in a wire support to keep the dosage form off of the bottom of the flask, so that all surfaces are exposed to the moving release solution and the solutions are stirred using paddles that rotate at a rate of 50 rpm. At each time interval, a single dosage form is removed from the solution, released material is removed from the surface, and the dosage form cut in half and placed in 100 mL of a recovery solution (1:1 wt/wt ethanol:water, pH adjusted to 3 with 0.1 N HCl), and vigorously stirred overnight at ambient temperature to dissolve the drug remaining in the dosage form. Samples of the recovery solution containing the dissolved drug are filtered using a Gelman Nylon® Acrodisc® 13, 0.45 μm pore size filter, and placed in a vial and capped. Residual drug is analyzed by HPLC. Drug concentration is calculated by comparing UV absorbance of samples to the absorbance of drug standards. The amount remaining in the tablets is subtracted from the total drug to obtain the amount released at each time interval.

An alternative in vitro test is a direct test, which is described below for sertraline HCl. Samples of the dosage form are placed into a stirred USP type 2 dissoette flask containing 900 mL of a receptor solution such as USP sodium acetate buffer (27 mM acetic acid and 36 mM sodium acetate, pH 4.5) or 88 mM NaCl. Samples are taken at periodic intervals using a VanKel VK8000 autosampling dissoette with automatic receptor solution replacement. Tablets are placed in a wire support as above, paddle height is adjusted, and the dissoette flasks stirred at 50 rpm at 37° C. The autosampler dissoette device is programmed to periodically remove a sample of the receptor solution, and the drug concentration is analyzed by HPLC using the procedure outlined above.

The sustained release core may be any solid dosage form core capable of being administered as a large core and coated with an enteric coating. Exemplary cores are matrix devices, osmotic cores and capsules.

### Matrix Devices

In one embodiment, the sustained release core is a matrix device in which the drug is incorporated into an erodible or non-erodible polymeric matrix. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or “matrix” that entraps the drug. The aqueous-swollen matrix gradually erodes, swells, disintegrates or dissolves in the environment of use, thereby controlling the release of drug to the environment of use. Examples of such dosage forms are well known in the art. See, for example, Remington: The Science and Practice of Pharmacy, 20^{th} Edition, 2000. Examples of such dosage forms are also disclosed in commonly assigned pending U.S. patent application Ser. No. 09/495,059 filed Jan. 31, 2000 which claimed the benefit of priority of provisional patent application Ser. No. 60/119,400 filed Feb. 10, 1999, the relevant disclosure of which is herein incorporated by reference. Other examples are disclosed in U.S. Pat. No. 4,839,177 and U.S. Pat. No. 5,484,608, herein incorporated by reference.

The erodible polymeric matrix into which drug is incorporated may generally be described as a set of excipients that are mixed with the drug that, when contacted with the aqueous environment of use imbibes water and forms a water-swollen gel or “matrix” that entraps the drug. Drug release may occur by a variety of mechanisms: the matrix may disintegrate or dissolve from around particles or granules of the drug; or the drug may dissolve in the imbibed aqueous solution and diffuse from the tablet, beads or granules of the dosage form. A key ingredient of this water-swollen matrix is the water-swellable, erodible, or soluble polymer, which may generally be described as an osmopolymer, hydrogel or water-swellable polymer. Such polymers may be linear, branched, or crosslinked. They may be homopolymers or copolymers. Although they may be synthetic polymers derived from vinyl, acrylate, methacrylate, urethane, ester and oxide monomers, they are most preferably derivatives of naturally occurring polymers such as polysaccharides or proteins. Exemplary materials include hydrophilic vinyl and acrylic polymers, polysaccharides such as calcium alginate, polyethylene oxide (PEO), polyethylene glycol (PEG), polypropylene glycol (PPG). Exemplary naturally occurring polymers include naturally occurring polysaccharides such as chitin, chitosan, dextran and pullulan; gum agar, gum arabic, gum karaya, locust bean gum, gum tragacanth, carrageenans, gum ghatti, guar gum, xanthan gum and scleroglucan; starches such as dextrin and maltodextrin; hydrophilic colloids such as pectin; phosphatides such as lecithin; alginates such as ammonium alginate, sodium, potassium or calcium alginate, propylene glycol alginate; gelatin; collagen; and cellulosics. By “cellulosics” is meant a cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent.

A preferred class of cellulosics for the erodible matrix comprises aqueous-soluble and aqueous-erodible cellulosics such as ethyl cellulose (EC), methylethyl cellulose (MEC), carboxymethyl cellulose (CMC), carboxymethyl ethylcellulose (CMEC), hydroxyethyl cellulose (HEC), hydroxypropyl cellulose (HPC), cellulose acetate (CA), cellulose propionate (CPr), cellulose butyrate (CB), cellulose acetate butyrate (CAB), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC). A particularly preferred class of such cellulosics comprises various grades of low viscosity (MW less than or equal to 50,000 daltons) and high viscosity (MW greater than 50,000 daltons) HPMC. Commercially available low viscosity HPMC polymers include the Dow METHOCEL series E5, E15LV, E50LV and K100LY, while high viscosity HPMC polymers include E4MCR, E10MCR, K4M, K15M and K100M; especially preferred in this group are the METHOCEL (Trademark) Kseries. Other commercially available types of HPMC include the Shin Etsu METOLOSE 90SH series.

Although the primary role of the erodible matrix material is to control the rate of release of drug to the environment of use, the inventors have found that the choice of matrix material can have a large effect on the maximum drug concentration attained by the dosage form as well as the maintenance of a high drug concentration. In one embodiment, the matrix material is a precipitation-inhibiting polymer, as defined herein.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

The erodible matrix polymer may also contain a wide variety of additives and excipients known in the pharmaceutical arts, including osmopolymers, osmagens, solubility-enhancing or -retarding agents and excipients that promote stability or processing of the dosage form.

Alternatively, the matrix device may be a non-erodible matrix dosage form. In such dosage forms, drug is distributed in an inert matrix. The drug is released by diffusion through the inert matrix. Examples of materials suitable for the inert matrix include insoluble plastics, such as methyl acrylate-methyl methacrylate copolymers, polyvinyl chloride, and polyethylene; hydrophilic polymers, such as ethyl cellulose, cellulose acetate, and crosslinked polyvinylpyrrolidone (also known as crospovidone); and fatty compounds, such as carnauba wax, microcrystalline wax, and triglycerides. Such dosage forms are described further in Remington: The Science and Practice of Pharmacy, 20^{th} edition (2000).

Matrix devices may be prepared by blending drug and other excipients together, and then forming the blend into a tablet, caplet, pill, or other dosage form formed by compressive forces. Such compressed dosage forms may be formed using any of a wide variety of presses used in the fabrication of pharmaceutical dosage forms. Examples include single-punch presses, rotary tablet presses, and multilayer rotary tablet presses, all well known in the art. See for example, Remington: The Science and Practice of Pharmacy, 20^{th} Edition, 2000. The compressed dosage form may be of any shape, including round, oval, oblong, cylindrical, or triangular. The upper and lower surfaces of the compressed dosage form may be flat, round, concave, or convex.

When formed by compression, the matrix device preferably has a “strength” of at least 5 Kiloponds (kp)/cm², and more preferably at least 7 kp/cm². Here, “strength” is the fracture force, also known as the tablet “hardness,” required to fracture a tablet formed from the materials, divided by the maximum cross-sectional area of the tablet normal to that force. The fracture force may be measured using a Schleuniger Tablet Hardness Tester, Model 6D. The compression force required to achieve this strength will depend on the size of the tablet, but generally will be greater than about 5 kp. Friability is a well-known measure of a dosage form's resistance to surface abrasion that measures weight loss in percentage after subjecting the dosage form to a standardized agitation procedure. Friability values of from 0.8 to 1.0% are regarded as constituting the upper limit of acceptability. Dosage forms having a strength of greater than about 5 kp/cm² generally are very robust, having a friability of less than about 0.5%.

Other methods for forming matrix devices forms are well known in the pharmaceutical arts. See for example, Remington: The Science and Practice of Pharmacy, 20^{th} Edition, 2000.

### Osmotic Cores

Alternatively, drug may be incorporated into an osmotic sustained release core. Such osmotic sustained release cores have at least two components: (a) an inner core which contains an osmotic agent and the drug; and (b) a water permeable, non-dissolving and non-eroding coating surrounding the inner core, the coating controlling the influx of water to the inner core from an aqueous environment of use so as to cause drug release by extrusion of some or all of the inner core to the environment of use. The osmotic agent contained in the inner core of the osmotic sustained release core may be an aqueous-swellable hydrophilic polymer or it may be an osmogen, also known as an osmagent. The coating is preferably polymeric, aqueous-permeable, and has at least one delivery port. Examples of such cores are well known in the art. See, for example, Remington: The Science and Practice of Pharmacy, 20^{th} Edition, 2000. Examples of such osmotic sustained release cores are also disclosed in U.S. Pat. No. 6,706,283, the relevant disclosure of which is herein incorporated by reference.

In addition to drug, the inner core of the osmotic sustained release core optionally includes an “osmotic agent.” By “osmotic agent” is meant any agent that creates a driving force for transport of water from the environment of use into the inner core. Exemplary osmotic agents are water-swellable hydrophilic polymers, and osmogens (or osmagens). Thus, the inner core may include water-swellable hydrophilic polymers, both ionic and nonionic, often referred to as “osmopolymers” and “hydrogels.” The amount of water-swellable hydrophilic polymers present in the inner core may range from about 5 to about 80 wt %, preferably 10 to 50 wt %. Exemplary materials include hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolate. Other materials include hydrogels comprising interpenetrating networks of polymers that may be formed by addition or by condensation polymerization, the components of which may comprise hydrophilic and hydrophobic monomers such as those just mentioned. Preferred polymers for use as the water-swellable hydrophilic polymers include PEO, PEG, PVP, sodium croscarmellose, HPMC, sodium starch glycolate, polyacrylic acid and crosslinked versions or mixtures thereof.

The inner core may also include an osmogen (or osmagent). The amount of osmogen present in the inner core may range from about 2 to about 70 wt %, preferably 10 to 50 wt %. Typical classes of suitable osmogens are water-soluble organic acids, salts and sugars that are capable of imbibing water to thereby effect an osmotic pressure gradient across the barrier of the surrounding coating. Typical useful osmogens include magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. Particularly preferred osmogens are glucose, lactose, sucrose, mannitol, xylitol and sodium chloride.

The inner core may include a wide variety of additives and excipients that enhance the performance of the inner core or that promote stability, tableting or processing. Such additives and excipients include tableting aids, surfactants, water-soluble polymers, pH modifiers, fillers, binders, pigments, disintegrants, antioxidants, lubricants and flavorants. Exemplary of such components are microcrystalline cellulose; metallic salts of acids such as aluminum stearate, calcium stearate, magnesium stearate, sodium stearate, and zinc stearate; pH control agents such as buffers, organic acids and organic acid salts and organic and inorganic bases; fatty acids, hydrocarbons and fatty alcohols such as stearic acid, palmitic acid, liquid paraffin, stearyl alcohol, and palmitol; fatty acid esters such as glyceryl (mono- and di-) stearates, triglycerides, glyceryl (palmiticstearic) ester, sorbitan esters, such as sorbitan monostearate, saccharose monostearate, saccharose monopalmitate, and sodium stearyl fumarate; polyoxyethylene sorbitan esters; surfactants, such as alkyl sulfates such as sodium lauryl sulfate and magnesium lauryl sulfate; polymers such as polyethylene glycols, polyoxyethylene glycols, polyoxyethylene and polyoxypropylene ethers and their copolymers, and polytetrafluoroethylene; and inorganic materials such as talc and dibasic calcium phosphate; cyclodextrins; sugars such as lactose and xylitol; and sodium starch glycolate. Examples of disintegrants are sodium starch glycolate (e.g., Explotab™), microcrystalline cellulose (e.g., Avicel™), microcrystalline silicified cellulose (e.g., ProSolv™), croscarmellose sodium (e.g., Ac-Di-Sol™).

One embodiment of an osmotic sustained release core consists of one or more drug layers containing drug, and a sweller layer that comprises a water-swellable polymer, with a coating surrounding the drug layer and sweller layer. Each layer may contain other excipients such as tableting aids, osmagents, surfactants, water-soluble polymers and water-swellable polymers.

Such osmotic sustained release cores may be fabricated in various geometries including bilayer, wherein the core comprises a drug layer and a sweller layer adjacent to each other; trilayer, wherein the inner core comprises a sweller layer “sandwiched” between two drug layers; and concentric, wherein the inner core comprises a central sweller composition surrounded by the drug layer.

The coating of such a tablet comprises a membrane permeable to water but substantially impermeable to drug and excipients contained within. The coating contains one or more exit passageways or ports in communication with the drug-containing layer(s) for delivering the drug composition. The drug-containing layer(s) of the inner core contains the drug composition (including optional osmagents and hydrophilic water-soluble polymers), while the sweller layer consists of an expandable hydrogel, with or without additional osmotic agents.

When placed in an aqueous medium, the osmotic sustained release core imbibes water through the coating surrounding the inner core, causing the composition to form a dispensable aqueous composition, and causing the hydrogel layer to expand and push against the drug-containing composition, forcing the composition out of the exit passageway. The composition can swell, aiding in forcing the drug out of the passageway. Drug can be delivered from this type of delivery system either dissolved or dispersed in the composition that is expelled from the exit passageway.

The rate of drug delivery is controlled by such factors as the permeability and thickness of the coating, the osmotic pressure of the drug-containing layer, the degree of hydrophilicity of the hydrogel layer, and the surface area of the inner core. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the inner core's surface area.

Exemplary materials useful in forming the drug-containing composition, in addition to drug, include HPMC, PEO and PVP and other pharmaceutically acceptable carriers. In addition, osmagents such as sugars or salts, especially sucrose, lactose, xylitol, mannitol, or sodium chloride, may be added. Materials which are useful for forming the hydrogel layer include sodium CMC, PEO, poly (acrylic acid), sodium (polyacrylate), sodium croscarmellose, sodium starch glycolate, PVP, crosslinked PVP, and other high molecular weight hydrophilic materials. Particularly useful are PEO polymers having an average molecular weight from about 5,000,000 to about 7,500,000 daltons.

In the case of a bilayer geometry, the delivery port(s) or exit passageway(s) may be located on the side of the tablet containing the drug composition or may be on both sides of the sustained release core or even on the edge of the tablet so as to connect both the drug layer and the sweller layer with the exterior of the inner core. The exit passageway(s) may be produced by mechanical means or by laser drilling, or by creating a difficult-to-coat region on the tablet by use of special tooling during tablet compression or by other means.

The osmotic sustained release core can also be made with a homogeneous inner core surrounded by a semipermeable membrane coating, as in U.S. Pat. No. 3,845,770. Drug can be incorporated into an inner core and a semipermeable membrane coating can be applied via conventional tablet-coating techniques such as using a pan coater. A drug delivery passageway can then be formed in this coating by drilling a hole in the coating, either by use of a laser or mechanical means. Alternatively, the passageway may be formed by rupturing a portion of the coating or by creating a region on the tablet that is difficult to coat, as described above.

A particularly useful embodiment of an osmotic core comprises: (a) a single-layer compressed inner core comprising: (i) drug, (ii) a hydroxyethylcellulose, and (iii) an osmagent, wherein the hydroxyethylcellulose is present in the core from about 2.0% to about 35% by weight and the osmagent is present from about 15% to about 70% by weight; (b) a water-permeable layer surrounding the inner core; and (c) at least one passageway within the layer (b) for delivering the drug to a fluid environment surrounding the tablet. Such inner cores are disclosed more fully in commonly owned, pending U.S. patent application Ser. No. 10/352,283, entitled “Osmotic Delivery System,” the disclosure of which are incorporated herein by reference.

Another example of an osmotic core is an osmotic capsule. The capsule shell or portion of the capsule shell can be semipermeable. The capsule can be filled either by a powder or liquid consisting of drug, excipients that imbibe water to provide osmotic potential, and/or a water-swellable polymer, or optionally solubilizing excipients. The capsule core can also be made such that it has a bilayer or multilayer composition analogous to the bilayer, trilayer or concentric geometries described above.

Another class of osmotic sustained release core useful in this invention comprises coated swellable tablets, as described in EP 378 404, incorporated herein by reference. Coated swellable tablets comprise a tablet inner core comprising the solubility-improved form of the drug and a swelling material, preferably a hydrophilic polymer, coated with a membrane, which contains holes, or pores through which, in the aqueous use environment, the hydrophilic polymer can extrude and carry out the drug composition. Alternatively, the membrane may contain polymeric or low molecular weight water-soluble “porosigens”. Porosigens dissolve in the aqueous use environment, providing pores through which the hydrophilic polymer and drug may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser, mechanical, or other means. In this class of osmotic sustained release cores, the coating material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release cores may also be multilayered, as described in EP 378 404 A2.

The osmotic sustained release cores of the present invention also comprise a coating. The essential constraints on the coating for an osmotic sustained release core are that it be water-permeable, have at least one port for the delivery of drug, and be non-dissolving and non-eroding during release of the drug formulation, such that drug is substantially entirely delivered through the delivery port(s) or pores as opposed to delivery primarily via permeation through the coating material itself. By “delivery port” is meant any passageway, opening or pore whether made mechanically, by laser drilling, by pore formation either during the coating process or in situ during use or by rupture during use. The coating should be present in an amount ranging from about 5 to 30 wt %, preferably 10 to 20 wt % relative to the core weight.

A preferred form of coating is a semipermeable polymeric membrane that has the port(s) formed therein either prior to or during use. Thickness of such a polymeric membrane may vary between about 20 and 800 μm, and is preferably in the range of 100 to 500 μm. The delivery port(s) should generally range in size from 0.1 to 3000 μm or greater, preferably on the order of 50 to 3000 μm in diameter. Such port(s) may be formed post-coating by mechanical or laser drilling or may be formed in situ by rupture of the coatings; such rupture may be controlled by intentionally incorporating a relatively small weak portion into the coating. Delivery ports may also be formed in situ by erosion of a plug of water-soluble material or by rupture of a thinner portion of the coating over an indentation in the core. In addition, delivery ports may be formed during coating, as in the case of asymmetric membrane coatings of the type disclosed in U.S. Pat. Nos. 5,612,059 and 5,698,220, the disclosures of which are incorporated by reference.

When the delivery port is formed in situ by rupture of the coating, a particularly preferred embodiment is a collection of beads that may be of essentially identical or of a variable composition. Drug is primarily released from such beads following rupture of the coating and, following rupture, such release may be gradual or relatively sudden. When the collection of beads has a variable composition, the composition may be chosen such that the beads rupture at various times following administration, resulting in the overall release of drug being sustained for a desired duration.

Coatings may be dense, microporous or “asymmetric,” having a dense region supported by a thick porous region such as those disclosed in U.S. Pat. Nos. 5,612,059 and 5,698,220. When the coating is dense the coating is composed of a water-permeable material. When the coating is porous, it may be composed of either a water-permeable or a water-impermeable material. When the coating is composed of a porous water-impermeable material, water permeates through the pores of the coating as either a liquid or a vapor.

Examples of osmotic cores that utilize dense coatings include U.S. Pat. Nos. 3,995,631 and 3,845,770, the disclosures of which pertaining to dense coatings are incorporated herein by reference. Such dense coatings are permeable to the external fluid such as water and may be composed of any of the materials mentioned in these patents as well as other water-permeable polymers known in the art.

The membranes may also be porous as disclosed in U.S. Pat. Nos. 5,654,005 and 5,458,887 or even be formed from water-resistant polymers. U.S. Pat. No. 5,120,548 describes another suitable process for forming coatings from a mixture of a water-insoluble polymer and a leachable water-soluble additive, the pertinent disclosures of which are incorporated herein by reference. The porous membranes may also be formed by the addition of pore-formers as disclosed in U.S. Pat. No. 4,612,008, the pertinent disclosures of which are incorporated herein by reference.

In addition, vapor-permeable coatings may even be formed from extremely hydrophobic materials such as polyethylene or polyvinylidene difluoride that, when dense, are essentially water-impermeable, as long as such coatings are porous.

Materials useful in forming the coating include various grades of acrylics, vinyls, ethers, polyamides, polyesters and cellulosic derivatives that are water-permeable and water-insoluble at physiologically relevant pHs, or are susceptible to being rendered water-insoluble by chemical alteration such as by crosslinking.

Specific examples of suitable polymers (or crosslinked versions) useful in forming the coating include plasticized, unplasticized and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxiated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes and synthetic waxes.

A preferred coating composition comprises a cellulosic polymer, in particular cellulose ethers, cellulose esters and cellulose ester-ethers, i.e., cellulosic derivatives having a mixture of ester and ether substituents.

Another preferred class of coating materials are poly(acrylic) acids and esters, poly(methacrylic) acids and esters, and copolymers thereof.

A more preferred coating composition comprises cellulose acetate. An even more preferred coating comprises a cellulosic polymer and PEG. A most preferred coating comprises cellulose acetate and PEG.

Coating is conducted in conventional fashion, typically by dissolving or suspending the coating material in a solvent and then coating by dipping, spray coating or preferably by pan-coating. A preferred coating solution contains 5 to 15 wt % polymer. Typical solvents useful with the cellulosic polymers mentioned above include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, water, and mixtures thereof. Pore-formers and non-solvents (such as water, glycerol and ethanol) or plasticizers (such as diethyl phthalate) may also be added in any amount as long as the polymer remains soluble at the spray temperature. Pore-formers and their use in fabricating coatings are described in U.S. Pat. No. 5,612,059, the pertinent disclosures of which are incorporated herein by reference.

Coatings may also be hydrophobic microporous layers wherein the pores are substantially filled with a gas and are not wetted by the aqueous medium but are permeable to water vapor, as disclosed in U.S. Pat. No. 5,798,119, the pertinent disclosures of which are incorporated herein by reference. Such hydrophobic but water-vapor permeable coatings are typically composed of hydrophobic polymers such as polyalkenes, polyacrylic acid derivatives, polyethers, polysulfones, polyethersulfones, polystyrenes, polyvinyl halides, polyvinyl esters and ethers, natural waxes and synthetic waxes. Especially preferred hydrophobic microporous coating materials include polystyrene, polysulfones, polyethersulfones, polyethylene, polypropylene, polyvinyl chloride, polyvinylidene fluoride and polytetrafluoroethylene. Such hydrophobic coatings can be made by known phase inversion methods using any of vapor-quench, liquid quench, thermal processes, leaching soluble material from the coating or by sintering coating particles. In thermal processes, a solution of polymer in a latent solvent is brought to liquid-liquid phase separation in a cooling step. When evaporation of the solvent is not prevented, the resulting membrane will typically be porous. Such coating processes may be conducted by the processes disclosed in U.S. Pat. Nos. 4,247,498; 4,490,431 and 4,744,906, the disclosures of which are also incorporated herein by reference.

Osmotic sustained-release cores may be prepared using procedures known in the pharmaceutical arts. See for example, Remington: The Science and Practice of Pharmacy, 20^{th} Edition, 2000.

### Capsules

As yet another embodiment, the sustained release core may comprise a sustained release capsule, such as an osmotic capsule. Osmotic capsules can be made using the same or similar components to those described above for osmotic cores. The capsule shell or portion of the capsule shell can be semipermeable and made of materials described above, and may be a hard gelatin capsule or a soft gelatin capsule, well known in the art (see, for example, Remington: The Science and Practice of Pharmacy, (20th ed. 2000). The capsule material may also consist of several layers, such as an outer enteric layer and an inner semipermeable layer. The capsule can be filled either by a powder or liquid consisting of the low-solubility drug, excipients that imbibe water to provide osmotic potential, and/or a water-swellable polymer, or optionally solubilizing excipients. The capsule core can also be made such that it has a bilayer or multilayer composition analogous to the bilayer, trilayer or concentric geometries described above. Sustained release capsules are further described in U.S. Pat. Nos. 4,627,850, 5,324,280, and 5,413,572, the disclosures of which are incorporated herein by reference.

### Precipitation-Inhibiting Polymers

In one embodiment, the dosage form also contains a precipitation-inhibiting polymer. Precipitation-inhibiting polymers suitable for use with the present invention should be inert, in the sense that they do not chemically react with drug in an adverse manner, are pharmaceutically acceptable, and have at least some solubility in aqueous solution at physiologically relevant pHs (e.g. 1-8). The polymer can be neutral or ionizable, and should have an aqueous-solubility of at least 0.1 mg/mL over at least a portion of the pH range of 1-8.

Precipitation-inhibiting polymers suitable for use with the present invention may be cellulosic or non-cellulosic. The polymers may be neutral or ionizable in aqueous solution. Of these, ionizable and cellulosic polymers are preferred, with ionizable cellulosic polymers being more preferred.

A preferred class of precipitation-inhibiting polymers comprises polymers that are “amphiphilic” in nature, meaning that the polymer has hydrophobic and hydrophilic portions. The hydrophobic portion may comprise groups such as aliphatic or aromatic hydrocarbon groups. The hydrophilic portion may comprise either ionizable or non-ionizable groups that are capable of hydrogen bonding such as hydroxyls, carboxylic acids, esters, amines or amides.

One class of precipitation-inhibiting polymers suitable for use with the present invention comprises neutral non-cellulosic polymers. Exemplary polymers include: vinyl polymers and copolymers having substituents of hydroxyl, alkylacyloxy, or cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyoxyethylene-polyoxypropylene copolymers, also known as poloxamers; and polyethylene polyvinyl alcohol copolymers.

Another class of precipitation-inhibiting polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGITS® manufactured by Rohm Tech Inc., of Malden, Mass.; amine-functionalized polyacrylates and polymethacrylates; proteins; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers, and polyoxyethylene-polyoxypropylene copolymers. Exemplary commercial grades of such copolymers include the EUDRAGITS, which are copolymers of methacrylates and acrylates, and the PLURONICS or LUTROLS supplied by BASF, which are polyoxyethylene-polyoxypropylene copolymers.

A preferred class of precipitation-inhibiting polymers comprises ionizable and neutral cellulosic polymers with at least one ester- and/or ether-linked substituent in which the polymer has a degree of substitution of at least 0.1 for each substituent.

It should be noted that in the polymer nomenclature used herein, ether-linked substituents are recited prior to “cellulose” as the moiety attached to the ether group; for example, “ethylbenzoic acid cellulose” has ethoxybenzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate groups attached via ester linkages to a significant fraction of the cellulosic polymer's hydroxyl groups. Generally, the degree of substitution of each substituent group can range from 0.1 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate substituted, the phthalate degree of substitution is 3. Also included within each polymer family type are cellulosic polymers that have additional substituents added in relatively small amounts that do not substantially alter the performance of the polymer.

Amphiphilic cellulosics comprise polymers in which the parent cellulosic polymer has been substituted at least a portion of the hydroxyl groups present on the saccharide repeat units of the polymer with at least one relatively hydrophobic substituent. Hydrophobic substituents may be essentially any substituent that, if substituted to a high enough level or degree of substitution, can render the cellulosic polymer essentially aqueous insoluble. Examples of hydrophobic substituents include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable substituents include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary non-ionizable polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

A preferred set of neutral cellulosic polymers are those that are amphiphilic. Exemplary polymers include hydroxypropyl methyl cellulose and hydroxypropyl methyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer. Neutral polymers suitable for use in the present invention are more fully disclosed in commonly assigned pending U.S. patent application Ser. No. 10/175,132, filed Jun. 18, 2002, herein incorporated by reference.

A preferred class of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic group may itself be ionizable, such as phthalate or trimellitate substituents.

Exemplary cellulosic polymers that are at least partially ionized at physiologically relevant pHs include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethyl ethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Exemplary cellulosic polymers that meet the definition of amphiphilic, having hydrophilic and hydrophobic regions include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

A particularly desirable subset of cellulosic ionizable polymers are those that possess both a carboxylic acid functional aromatic substituent and an alkylate substituent and thus are amphiphilic. Exemplary polymers include cellulose acetate phthalate, methyl cellulose acetate phthalate, ethyl cellulose acetate phthalate, hydroxypropyl cellulose acetate phthalate, hydroxypropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another particularly desirable subset of cellulosic ionizable polymers are those that possess a non-aromatic carboxylate substituent. Exemplary polymers include hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate, and carboxymethyl ethyl cellulose.

While, as listed above, a wide range of polymers may be used, the inventors have found that relatively hydrophobic polymers have shown the best performance as demonstrated in vitro dissolution tests. In particular, cellulosic polymers that are aqueous insoluble in their nonionized state but are aqueous soluble in their ionized state perform particularly well. A particular subclass of such polymers are the so-called “enteric” polymers, which include, for example, hydroxypropyl methyl cellulose acetate succinate (HPMCAS), hydroxypropyl methyl cellulose phthalate (HPMCP), cellulose acetate phthalate (CAP), cellulose acetate trimellitate (CAT), and carboxymethyl ethyl cellulose (CMEC). In addition, non-enteric grades of such polymers, as well as closely related cellulosic polymers, are expected to perform well due to the similarities in physical properties.

Thus, especially preferred polymers are hydroxypropyl methyl cellulose acetate succinate (HPMCAS), hydroxypropyl methyl cellulose phthalate (HPMCP), cellulose acetate phthalate (CAP), cellulose acetate trimellitate (CAT), methyl cellulose acetate phthalate, hydroxypropyl methyl cellulose acetate phthalate, cellulose acetate terephthalate, cellulose acetate isophthalate, and carboxymethyl ethyl cellulose (CMEC). The most preferred ionizable cellulosic polymers are hydroxypropyl methyl cellulose acetate succinate, hydroxypropyl methyl cellulose phthalate, cellulose acetate phthalate, cellulose acetate trimellitate, and carboxymethyl ethyl cellulose.

While specific polymers have been discussed as being suitable for use in the dosage forms of the present invention, blends of such polymers may also be suitable. Thus the term “polymer” is intended to include blends of polymers in addition to a single species of polymer.

Another preferred class of polymers consists of neutralized acidic polymers. By “neutralized acidic polymer” is meant any acidic polymer for which a significant fraction of the “acidic moieties” or “acidic substituents” have been “neutralized”; that is, exist in their deprotonated form. By “acidic polymer” is meant any polymer that possesses a significant number of acidic moieties. In general, a significant number of acidic moieties would be greater than or equal to about 0.1 milliequivalents of acidic moieties per gram of polymer. “Acidic moieties” include any functional groups that are sufficiently acidic that, in contact with or dissolved in water, can at least partially donate a hydrogen cation to water and thus increase the hydrogen-ion concentration. This definition includes any functional group or “substituent,” as it is termed when the functional group is covalently attached to a polymer that has a pKa of less than about 10. Exemplary classes of functional groups that are included in the above description include carboxylic acids, thiocarboxylic acids, phosphates, phenolic groups, and sulfonates. Such functional groups may make up the primary structure of the polymer such as for polyacrylic acid, but more generally are covalently attached to the backbone of the parent polymer and thus are termed “substituents.” Neutralized acidic polymers are described in more detail in commonly assigned copending U.S. patent application Ser. No. 10/175,566 entitled “Pharmaceutical Compositions of Drugs and Neutralized Acidic Polymers” filed Jun. 17, 2002, the relevant disclosure of which is incorporated by reference.

In addition, the preferred polymers listed above, that is amphiphilic cellulosic polymers, tend to have greater precipitation-inhibiting properties relative to the other polymers of the present invention. Generally those precipitation-inhibiting polymers that have ionizable substituents tend to perform best. In vitro tests of compositions with such polymers tend to have higher MDC and AUC values than compositions with other polymers of the invention.

The precipitation-inhibiting polymer is present in a sufficient amount to improve the concentration of dissolved drug relative to the low-solubility drug alone (that is, drug in solubility-improved form but no precipitation-inhibiting polymer). Several methods, such as an in vitro dissolution test or a membrane permeation test may be used to evaluate precipitation-inhibiting polymers and the degree of concentration enhancement provided by the polymers. It has been determined that enhanced drug concentration in in vitro dissolution tests in MFD solution, PBS solution, or simulated intestinal buffer solution, is a good indicator of in vivo performance and bioavailability. When tested using an in vitro dissolution test described above, the composition of low-solubility drug and precipitation-inhibiting polymer meets at least one, and preferably both, of the following conditions. The first condition is that the composition increases the maximum dissolved drug concentration (MDC) of drug in the in vitro dissolution test relative to a control composition consisting of an equivalent amount of drug in the solubility-improved form but no polymer. That is, once the composition is introduced into an environment of use, the composition provides an increased aqueous MDC of drug relative to the control composition. The control composition consists of the solubility-improved form of drug alone (without the precipitation-inhibiting polymer). Preferably, the inventive compositions provide an MDC of drug in aqueous solution that is at least 1.25-fold that of the control composition, more preferably at least 2-fold, and most preferably at least 3-fold. For example, if the MDC provided by the test composition is 5 mg/ml, and the MDC provided by the control composition is 1 mg/ml, the test composition provides an MDC that is fold that provided by the control composition.

The second condition is that the composition of low-solubility drug and polymer provides an increased dissolution area under the concentration versus time curve (AUC) of drug in the in vitro dissolution test relative to a control composition consisting of an equivalent amount of the drug in solubility-improved form but no polymer. (The calculation of an AUC is a well-known procedure in the pharmaceutical arts and is described, for example, in Welling, “Pharmacokinetics Processes and Mathematics,” ACS Monograph 185 (1986).) More specifically, in the environment of use, the composition of low-solubility drug and polymer provides an AUC for any 90-minute period of from about 0 to about 270 minutes following introduction to the use environment that is at least 1.25-fold that of the control composition described above. Preferably, the AUC provided by the composition is at least 2-fold, more preferably at least 3-fold that of the control composition.

A typical in vitro test to evaluate enhanced drug concentration in aqueous solution can be conducted by (1) adding with agitation a sufficient quantity of control composition, that is, the solubility-improved form of the drug alone, to the in vitro test medium, such as an MFD, PBS, or simulated intestinal buffer solution, to achieve equilibrium concentration of drug; (2) in a separate test, adding with agitation a sufficient quantity of test composition (e.g., the composition comprising the combination of the drug in a solubility-improved form and the precipitation-inhibiting polymer) in the same test medium, such that if all drug dissolved, the theoretical concentration of drug would exceed the equilibrium concentration of drug by a factor of at least 2, and preferably by a factor of at least 10; and (3) comparing the measured MDC and/or aqueous AUC of the test composition in the test medium with the equilibrium concentration, and/or with the aqueous AUC of the control composition. In conducting such a dissolution test, the amount of test composition or control composition used is an amount such that if all of the drug dissolved, the drug concentration would be at least 2-fold, preferably at least 10-fold, and most preferably at least 100-fold that of the equilibrium concentration.

Alternatively, an in vitro membrane-permeation test may also be used to determine if the composition comprising the precipitation-inhibiting polymer provides concentration enhancement relative to the control composition. In this test, described above, the composition is placed in, dissolved in, suspended in, or otherwise delivered to the aqueous solution to form a feed solution. A typical in vitro membrane-permeation test to evaluate the compositions of the invention can be conducted by (1) administering a sufficient quantity of test composition (that is, the solubility-improved drug form with precipitation-inhibiting polymer) to a feed solution, such that if all of the drug dissolved, the theoretical concentration of drug would exceed the equilibrium concentration of the drug by a factor of at least 2; (2) in a separate test, adding an equivalent amount of control composition (that is, the solubility-improved form of the drug alone) to an equivalent amount of test medium; and (3) determining whether the measured maximum flux of drug provided by the test composition is at least 1.25-fold that provided by the control composition. The solubility-improved form and precipitation-inhibiting polymer, when dosed to an aqueous use environment, provide a maximum flux of drug in the above test that is at least about 1.25-fold the maximum flux provided by the control composition. Preferably, the maximum flux provided by the test composition is at least about 1.5-fold, more preferably at least about 2-fold, and even more preferably at least about 3-fold that provided by the control composition.

The sustained-release cores of this embodiment comprise a combination of a solubility-improved form of drug and a precipitation-inhibiting polymer. “Combination” as used herein means that the solubility-improved form and precipitation-inhibiting polymer may be in physical contact with each other or in close proximity but without the necessity of being physically mixed. For example, wherein the sustained release core comprises multiple layers, one or more layers may comprise the solubility-improved form and one or more different layers comprises the precipitation-inhibiting polymer. Yet another example may constitute a coated core wherein either the solubility-improved form of the drug or the precipitation-inhibiting polymer or both may be present in the core and the coating may comprise the solubility-improved form or the precipitation-inhibiting polymer or both. Alternatively, the combination can be in the form of a simple dry physical mixture wherein both the solubility-improved form and precipitation-inhibiting polymer are mixed in particulate form and wherein the particles of each, regardless of size, retain the same individual physical properties that they exhibit in bulk. Any conventional method used to mix the polymer and drug together such as physical mixing and dry or wet granulation, may be used.

The combination of solubility-improved form and the precipitation-inhibiting polymer may be prepared by dry- or wet-mixing the drug or drug mixture with the precipitation-inhibiting polymer to form the composition. Mixing processes include physical processing as well as wet-granulation and coating processes.

For example, mixing methods include convective mixing, shear mixing, or diffusive mixing. Convective mixing involves moving a relatively large mass of material from one part of a powder bed to another, by means of blades or paddles, revolving screw, or an inversion of the powder bed. Shear mixing occurs when slip planes are formed in the material to be mixed. Diffusive mixing involves an exchange of position by single particles. These mixing processes can be performed using equipment in batch or continuous mode. Tumbling mixers (e.g., twin-shell) are commonly used equipment for batch processing. Continuous mixing can be used to improve composition uniformity.

Milling may also be employed to prepare the compositions of the present invention. Milling is the mechanical process of reducing the particle size of solids (comminution). Because in some cases milling may alter crystalline structure and cause chemical changes for some materials, milling conditions are generally chosen which do not alter the physical form of the drug. The most common types of milling equipment are the rotary cutter, the hammer, the roller and fluid energy mills. Equipment choice depends on the characteristics of the ingredients in the drug form (e.g., soft, abrasive, or friable). Wet- or dry-milling techniques can be chosen for several of these processes, also depending on the characteristics of the ingredients (e.g. drug stability in solvent). The milling process may serve simultaneously as a mixing process if the feed materials are heterogeneous. Conventional mixing and milling processes suitable for use in the present invention are discussed more fully in Lachman, et al., The Theory and Practice of Industrial Pharmacy (3d Ed. 1986). The components of the compositions of this invention may also be combined by dry- or wet-granulating processes.

In one preferred embodiment, the combination comprises particles of the solubility-improved form of drug at least partially coated with a precipitation-inhibiting polymer. The particles may be either drug crystals, or particles of some other solubility-improved form such as amorphous drug or a cyclodextrin complex. This embodiment finds particularly utility when it is desired to provide absorption of drug in the intestine, particularly the colon. Without wishing to be bound by theory, when the polymer and drug are released into the intestinal use environment, the polymer may begin to dissolve and gel prior to dissolution of the drug. Thus, as the drug dissolves into the intestinal use environment, the dissolved drug immediately encounters dissolved polymer surrounding the dissolved drug. This has the advantage of preventing nucleation of the drug, thus reducing the rate of precipitation of the drug.

The polymer may be coated around the drug crystals using any conventional method. A preferred method is a spray drying process. The term spray-drying is used conventionally and broadly refers to processes involving breaking up liquid mixtures or suspensions into small droplets (atomization) and rapidly removing solvent from the droplets in a container where there is a strong driving force for evaporation of solvent.

To coat the drug particles by spray drying, first a suspension of drug particles and dissolved polymer is formed in a solvent. The relative amounts of drug suspended in the solvent and polymer dissolved in the solvent are chosen to yield the desired drug to polymer ratio in the resulting particles. For example, if a particle having a drug to polymer ratio of 0.33 (25 wt % drug) is desired, then the spray solution comprises 1 part drug particles and 3 parts polymer dissolved in the solvent. The total solids content of the spray solution is preferably sufficiently high so that the spray solution results in efficient production of the particles. The total solids content refers to the amount of solid drug, dissolved polymer and other excipients dissolved in the solvent. For example, to form a spray solution having a 5 wt % dissolved solids content and which results in a particle having a 25 wt % drug loading, the spray solution would comprise 1.25 wt % drug, 3.75 wt % polymer and 95 wt % solvent. To achieve good yield, the spray solution preferably has a solids content of at least 3 wt %, more preferably at least 5 wt %, and even more preferably at least 10 wt %. However, the dissolved solids content should not be too high, or else the spray solution may be too viscous to atomize efficiently into small droplets.

The solvent is chosen based on the following characteristics: (1) the drug is insoluble or only slightly soluble in the solvent, (2) the polymer is soluble in the solvent; and (3) the solvent is relatively volatile. Preferably, the solubility of the drug in the solvent is less than 5 wt % of the amount of drug suspended in the spray solution, more preferably less than 1 wt % of the amount of drug suspended in the spray solution, and even more preferably less than 0.5 wt % of the amount of drug suspended in the spray solution. For example, if the spray solution contains 10 wt % drug, the drug preferably has a solubility of less than 0.5 wt %, more preferably less than 0.1 wt %, and even more preferably less than 0.05 wt % in the solvent. Preferred solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, THF, cyclic ethers, and 1,1,1-trichloroethane. Mixtures of solvents, may also be used, as can mixtures with water as long as the polymer is sufficiently soluble to make the spray-drying process practicable and as long as the drug is sufficiently insoluble to remain in suspension and not dissolved. In some cases it may be desired to add a small amount of water to aid solubility of the polymer in the spray solution.

Spray drying to form polymer coatings around drug particles is well known and is described in, for example, U.S. Pat. No. 4,767,789, U.S. Pat. No. 5,013,537, and U.S. published patent application 2002/0064108A1, herein incorporated by reference.

Alternatively, the polymer may be coated around the drug crystals using a rotary disk atomizer, as described in U.S. Pat. No. 4,675,140, herein incorporated by reference.

Alternatively, the precipitation-inhibiting polymer may be sprayed onto the drug particles in a high shear mixer or a fluid bed.

The amount of precipitation-inhibiting polymer may vary widely. In general, the amount of precipitation-inhibiting polymer should be sufficient to provide concentration-enhancement of the drug relative to a control composition consisting of the drug alone as described above. The weight ratio of solubility-improved form to precipitation-inhibiting polymer may range from 0.01 to 100. Good results are generally achieved where the polymer to drug weight ratio is at least 0.33 (at least 25 wt % polymer), more preferably at least 0.66 (at least 40 wt % polymer), and even more preferably at least 1 (at least 50 wt % polymer). However, since it is desired to limit the size of the dosage form, the amount of precipitation-inhibiting polymer may be less than the amount that provides the greatest degree of concentration enhancement.

### Enteric Coating

The sustained release core is coated with an enteric coating so that the sustained release core does not begin to release drug, or at least a substantial portion of the drug in the sustained release core, in the stomach. By “enteric coating” is meant an acid resistant coating that remains intact and does not dissolve at pH of less than about 4. The enteric coating surrounds the sustained release core so that the core does not dissolve or erode in the stomach, and the sustained release core does not begin to release substantial amounts of drug. The enteric coating may include an enteric coating polymer. Enteric coating polymers are generally polyacids having a pKₐ of about 3 to 5. Examples of enteric coating polymers include: cellulose derivatives, such as cellulose acetate phthalate, cellulose acetate trimellitate, hydroxypropyl methyl cellulose acetate succinate, cellulose acetate succinate, carboxy methyl ethyl cellulose, methylcellulose phthalate, and ethylhydroxy cellulose phthalate; vinyl polymers, such as polyvinyl acetate phthalate, vinyl acetate-maleic anhydride copolymer; polyacrylates; and polymethacrylates such as methyl acrylate-methacrylic acid copolymer, methacrylate-methacrylic acid-octyl acrylate copolymer; and styrene-maleic mono-ester copolymer. These may be used either alone or in combination, or together with other polymers than those mentioned above.

One class of preferred coating materials are the pharmaceutically acceptable methacrylic acid copolymer which are copolymers, anionic in character, based on methacrylic acid and methyl methacrylate, for example having a ratio of free carboxyl groups:methyl-esterified carboxyl groups of 1:3, e.g. around 1:1 or 1:2, and with a mean molecular weight of 135,000 daltons. Some of these polymers are known and sold as enteric polymers, for example having a solubility in aqueous media at pH 5.5 and above, such as the commercially available EUDRAGIT enteric polymers, such as Eudragit L 30, a cationic polymer synthesized from dimethylaminoethyl methacrylate, Eudragit S and Eudragit NE.

The coating may include conventional plasticizers, including dibutyl phthalate; dibutyl sebacate; diethyl phthalate; dimethyl phthalate; triethyl citrate; benzyl benzoate; butyl and glycol esters of fatty acids; mineral oil; oleic acid; stearic acid; cetyl alcohol; stearyl alcohol; castor oil; corn oil; coconut oil; and camphor oil; and other excipients such as anti-tack agents, glidants, etc. For plasticizers, triethyl citrate, coconut oil and dibutyl sebacate are particularly preferred. Typically the coating may include from about 0.1 to about 25 wt % plasticizer and from about 0.1 to about 10 wt % anti-tack agent.

The enteric coating may also include insoluble materials, such as alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin, 1,2-, 3,4-diepoxybutane, etc. The enteric coating may also include starch and/or dextrin.

The enteric coating may be applied to the sustained release core by dissolving or suspending the enteric coating materials in a suitable solvent. Examples of solvents suitable for use in applying a coating include alcohols, such as methanol, ethanol, isomers of propanol and isomers of butanol; ketones, such as acetone, methylethyl ketone and methyl isobutyl ketone; hydrocarbons, such as pentane, hexane, heptane, cyclohexane, methylcyclohexane, and octane; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methylpyrrolidinone; acetonitrile; water; and mixtures thereof.

Coating may be conducted by conventional techniques, such as by pan coaters, rotary granulators and fluidized bed coaters such as top-spray, tangential-spray or bottom-spray (Würster coating).

One preferred coating solution consists of about 40 wt % Eudragit L30-D55 and 2.5 wt % triethylcitrate in about 57.5 wt % water. This enteric coating solution may be coated onto the core using a pan coater or other suitable coating methods previously described.

### Immediate Release

The controlled release dosage form also comprises an immediate release portion comprising the low-solubility drug. By “immediate release portion” is meant broadly that a portion of the drug may be released within the two hours or less following administration. “Administration” to a use environment means, where the in vivo use environment is the GI tract, delivery by ingestion or swallowing or other such means to deliver the dosage form. Where the use environment is in vitro, “administration” refers to placement or delivery of the dosage form to the in vitro test medium. The dosage form may release at least 70 wt % of the drug initially present in the immediate release portion of the dosage form within two hours or less following introduction to a gastric use environment. Preferably, the dosage form releases at least 80 wt % during the first two hours, and most preferably, at least 90 wt % of the drug initially in the immediate release portion of the dosage form during the first two hours after administering the dosage form to a use environment. Immediate release of drug may be accomplished by any means known in the pharmaceutical arts, including immediate release coatings, layers, powders, multiparticulates or granules.

Virtually any means for providing immediate release of the drug known in the pharmaceutical arts can be used with the dosage form of the present invention. In one embodiment, the drug in the immediate release portion is in the form of an immediate release coating that surrounds the enteric coated sustained release core. The drug in the immediate release portion may be combined with a water soluble or water dispersible polymer, such as HPC, HPMC, HEC, PVP, and the like. The coating can be formed using solvent-based coating processes, powder-coating processes, and hot-melt coating processes, all well known in the art. In solvent-based processes, the coating is made by first forming a solution or suspension comprising the solvent, the drug, the coating polymer and optional coating additives. Preferably, the drug is suspended in the coating solvent. The coating materials may be completely dissolved in the coating solvent, or only dispersed in the solvent as an emulsion or suspension or anywhere in between. Latex dispersions, including aqueous latex dispersions, are a specific example of an emulsion or suspension that may be useful as a coating solution. The solvent used for the solution should be inert in the sense that it does not react with or degrade the drug, and be pharmaceutically acceptable. In one aspect, the solvent is a liquid at room temperature. Preferably, the solvent is a volatile solvent. By “volatile solvent” is meant that the material has a boiling point of less than about 150° C. at ambient pressure, although small amounts of solvents with higher boiling points can be used and acceptable results still obtained.

Examples of solvents suitable for use in applying a coating to an enteric coated sustained release core include alcohols, such as methanol, ethanol, isomers of propanol and isomers of butanol; ketones, such as acetone, methylethyl ketone and methyl isobutyl ketone; hydrocarbons, such as pentane, hexane, heptane, cyclohexane, methylcyclohexane, octane and mineral oil; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methylpyrrolidinone; acetonitrile; water; and mixtures thereof.

The coating formulation may also include additives to promote the desired immediate release characteristics or to ease the application or improve the durability or stability of the coating. Types of additives include plasticizers, pore formers, and glidants. Examples of coating additives suitable for use in the compositions of the present invention include plasticizers, such as mineral oils, petrolatum, lanolin alcohols, polyethylene glycol, polypropylene glycol, triethyl citrate, sorbitol, triethanol amine, diethyl phthalate, dibutyl phthalate, castor oil, triacetin and others known in the art; emulsifiers, such as polysorbate-80; pore formers, such as polyethylene glycol, polyvinyl pyrrolidone, polyethylene oxide, hydroxyethyl cellulose and hydroxypropylmethyl cellulose; and glidants, such as colloidal silicon dioxide, talc and cornstarch. In one embodiment, the drug is suspended in a commercially available coating formulation, such as Opadry® clear (available from Colorcon, Inc., WestPoint, Pa.). Coating is conducted in conventional fashion, typically by dipping, fluid-bed coating, spray-coating, or pan-coating.

The immediate release coating may also be applied using powder coating techniques well known in the art. In these techniques, the drug is blended with optional coating excipients and additives, to form an immediate release coating composition. This composition may then be applied using compression forces, such as in a tablet press.

The coating may also be applied using a hot-melt coating technique. In this method, a molten mixture comprising the drug and optional coating excipients and additives, is formed and then sprayed onto the enteric coated sustained release core. Typically, the hot-melt coating is applied in a fluidized bed equipped with a top-spray arrangement.

In another embodiment, the immediate release portion is first formed into an immediate release powder, multiparticulates or granules that are combined with the enteric coated sustained release core. The immediate release powder, multiparticulates, or granules may be combined with the enteric coated sustained release core in a capsule. In one aspect, the immediate-release composition consists essentially of the drug. In another aspect, the immediate-release composition comprises optional excipients, such as binders, stabilizing agents, diluents, disintegrants, and surfactants. Such immediate release powders may be formed by any conventional method for combining the drug and excipients. Exemplary methods include wet and dry granulation.

In addition to the drug, the immediate release portion may include other excipients to aid in formulating the immediate release portion. See, for example, Remington: The Science and Practice of Pharmacy (20th ed. 2000). Examples of other excipients include disintegrants, porosigens, matrix materials, fillers, diluents, lubricants, glidants, and the like, such as those previously described.

### Exemplary Embodiments

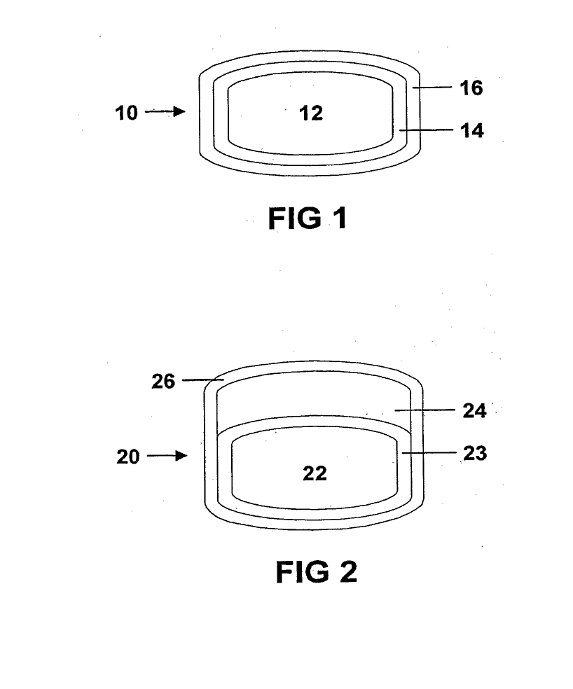

In one embodiment, the dosage form comprises an immediate release portion and an enteric coated sustained release core, wherein the sustained release core is in the form of a matrix controlled release device and the immediate release portion is in the form of an immediate release coating. Referring to FIG. 1, in one aspect, the dosage form 10 is in the form of a matrix tablet 12 comprising the drug (optionally in solubility-improved form) that is coated with an enteric coating 14. An immediate release coating 16 comprising the drug and optional excipients, as discussed above surrounds the enteric coating 14. The immediate release coating 16 may optionally be coated with a conventional coating (not shown in FIG. 1).

Alternatively, the dosage form comprises an immediate release portion and an enteric coated sustained release core, shown schematically as dosage form 20 in FIG. 2. The sustained release core 22 is in the form of a matrix controlled release device coated with an enteric coating 23, and the immediate release portion is in the form of an immediate release layer 24 associated with the matrix device. By associated with is meant that the immediate release layer comprising the drug 24 is adjacent to or substantially in contact with the enteric coated matrix controlled release device 22. The immediate release layer 24 may also be separated from the matrix controlled-release device by an intermediate layer (not shown in FIG. 2) comprising a binder or diluent, as known in the art. The dosage form 20 may optionally be coated with a conventional coating 26.

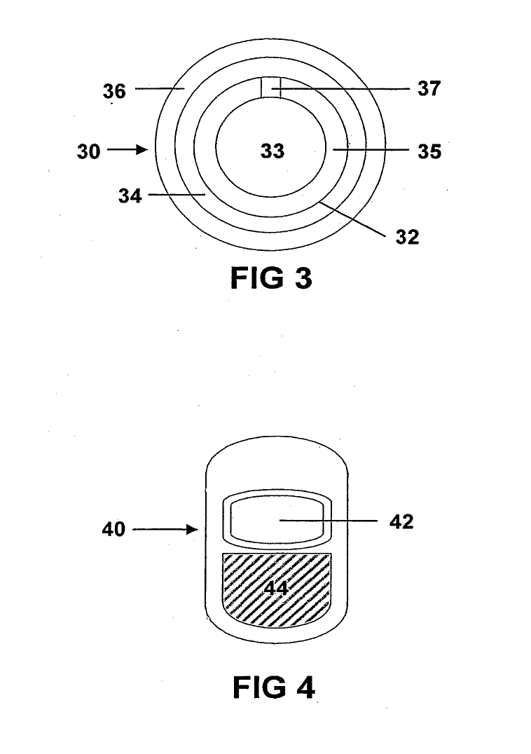

In another embodiment, the dosage form comprises an immediate release portion and an enteric coated sustained release core, shown schematically as dosage form 30 in FIG. 3. The sustained release core is in the form of an osmotic controlled release device 32 having an enteric coating 34 and the immediate release portion is in the form of an immediate release coating 36. The osmotic controlled release device 32 comprises a core 33, a coating 35, and a delivery port 37. The core may be a single composition, or may consist of several layers, including layers comprising the drug in solubility-improved form and highly swelling layers for extruding the drug into the use environment. The immediate release coating 36 may optionally be coated with a conventional coating (not shown in FIG. 3).

In another embodiment, the dosage form is in the form of a capsule, the capsule, shown schematically as dosage form 40 in FIG. 4. The capsule comprises (1) at least one enteric coated sustained-release device 42, such as a matrix controlled release device or an osmotic controlled release device, comprising the drug (optionally in solubility-improved form), and (2) an immediate release composition 44. In this embodiment, the sustained release device 42 comprising the drug and the immediate release composition 44 are first made using procedures known in the art, and then may be combined, such as by placing into a suitable capsule, such as a hard gelatin capsule or a soft gelatin capsule, well known in the art (see, for example, Remington: The Science and Practice of Pharmacy, (20th ed. 2000)). In one embodiment, the sustained release core is in the form of a matrix controlled-release device previously discussed. In another embodiment, the sustained release core is in the form of an osmotic controlled-release device, previously discussed. The immediate release composition 44 may be simply particles of the active drug alone, or it may be combined with optional excipients such that it is in the form of a powder, granules, or multiparticulates, previously described.

### Amounts of Drug and Administration

The relative amount of drug in the immediate release portion and the sustained release portion may be as desired in order to obtain desired blood levels of drug. In exemplary embodiments, the immediate release layer may contain from about 20 to 80 wt % of the active drug in the dosage form, while the sustained release core may contain from about 80 wt % to about 20 wt % of the active drug in the dosage form.

The dosage forms are administered orally. The dosage forms are preferably administered in the fed state in order to maximize gastric retention of the dosage form so as to increase the time during which the concentration of drug in the blood (serum or plasma) is greater than the therapeutically effective concentration of drug. The inventors have found that, in general, gastric retention times when administered in the fed state for large cores range from about 2 to 8 hours; in contrast, gastric retention times when administered in the fasted state range from about 0.5 to about 2 hours.

Other features and embodiments of the invention will become apparent from the following examples that are given for illustration of the invention rather than for limiting its intended scope.

### EXAMPLES

### Solubility-Improved Forms of Ziprasidone

Microcentrifuge dissolution tests were performed to evaluate the hydrochloride crystalline salt form of ziprasidone to verify that it was a solubility-improved form of ziprasidone. For this test, a sufficient amount of ziprasidone hydrochloride monohydrate was added to a microcentrifuge test tube so that the concentration of ziprasidone would have been 200 μgA/mL, if all of the ziprasidone had dissolved. The tests were run in duplicate. The tubes were placed in a 37° C. temperature-controlled chamber, and 1.8 mL MFD solution at pH 6.5 and 290 mOsm/kg was added to each respective tube. The samples were quickly mixed using a vortex mixer for about 60 seconds. The samples were centrifuged at 13,000 G at 37° C. for 1 minute prior to collecting a sample. The resulting supernatant solution was then sampled and diluted 1:5 (by volume) with methanol. Samples were analyzed by high-performance liquid chromatography (HPLC) at a UV absorbance of 315 nm using a Zorbax RxC8 Reliance column and a mobile phase consisting of 55% (50 mM potassium dihydrogen phosphate, pH 6.5)/45% acetonitrile. Drug concentration was calculated by comparing UV absorbance of samples to the absorbance of drug standards. The contents of each tube were mixed on the vortex mixer and allowed to stand undisturbed at 37° C. until the next sample was taken. Samples were collected at 4, 10, 20, 40, 90, and 1200 minutes following administration to the MFD solution. The results are shown in Table 1.

A similar test was performed with the crystalline ziprasidone free base as a control, and a sufficient amount of material was added so that the concentration of compound would have been 200 μgA/mL, if all of the ziprasidone had dissolved.

TABLE 1

| | | Dissolved Ziprasidone | AUC |
|---|---|---|---|
| Salt Form | Time (min) | Concentration (μgA/mL) | (min-μgA/mL) |
| Ziprasidone | 0 | 0 | 0 |
| Free Base | 4 | 1 | 3 |
| | 10 | 1 | 11 |
| | 20 | 1 | 23 |
| | 40 | 2 | 51 |
| | 90 | 1 | 120 |
| | 1200 | 2 | 2000 |
| Ziprasidone | 0 | 0 | 0 |
| hydrochloride | 4 | 14 | 30 |
| monohydrate | 10 | 15 | 110 |
| | 20 | 20 | 280 |
| | 40 | 22 | 700 |
| | 90 | 18 | 1,700 |
| | 1200 | 9 | 16,400 |

The concentrations of ziprasidone obtained in these tests were used to determine the maximum dissolved concentration of ziprasidone (“MDC₉₀”) and the area under the concentration-versus-time curve (“AUC₉₀”) during the initial ninety minutes. The results are shown in Table 2.

TABLE 2

| | MDC₉₀ | AUC₉₀ |
|---|---|---|
| Salt Form | (mgA/mL) | (min*mgA/mL) |
| Ziprasidone Free Base | 2 | 120 |
| Ziprasidone hydrochloride | 22 | 1,700 |
| monohydrate | | |

These results show that ziprasidone hydrochloride monohydrate provided an MDC₉₀ that was 11-fold that provided by the free base, and an AUC₉₀ that was 14-fold that provided by the free base. Thus, the hydrochloride salt form is a solubility-improved form of ziprasidone.

Ziprasidone Crystals Coated with Precipitation-Inhibiting Polymers

Ziprasidone coated crystals comprising 35% active ziprasidone hydrochloride monohydrate coated with the precipitation-inhibiting polymer, HPMCAS-H, were prepared as follows. A spray suspension was first formed by dissolving HPMCAS (AQOAT HG grade from Shin Etsu, Tokyo, Japan) in acetone in a container equipped with a top-mounted mixer. Crystalline particles of ziprasidone hydrochloride monohydrate, having a mean particle size of about 10 μm, were then added to the polymer solution and mixing continued with a top-mounted mixer. The composition consisted of 3.97 wt % crystalline ziprasidone hydrochloride monohydrate particles suspended in 6.03 wt % HPMCAS, and 90 wt % acetone. Next, a re-circulation pump (Yamada air actuated diaphragm pump model NDP-5FST) was used to transfer the suspension to a high-shear in line mixer (Bematek model LZ-150-6-PB multi-shear in-line mixer) where a series of rotor/stator shear heads broke up any remaining drug crystal agglomerations. The high shear mixer was operated with a setting of 3500±500 rpm, for 45-60 minutes per 20 kg solution. The re-circulation pump pressure was 35±10 psig.

The suspension was then pumped using a high-pressure pump to a spray drier (a Niro type XP Portable Spray-Dryer with a Liquid-Feed Process Vessel (“PSD-1”)), equipped with a pressure nozzle (Spraying Systems Pressure Nozzle and Body-SK 74-20). The PSD-1 was equipped with a 5-foot 9-inch chamber extension. The chamber extension was added to the spray dryer to increase the vertical length of the dryer. The added length increased the residence time within the dryer, which allowed the product to dry before reaching the angled section of the spray dryer. The spray drier was also equipped with a 316 stainless steel circular diffuser plate with 1/16-inch drilled holes, having a 1% open area. This small open area directed the flow of the drying gas to minimize product recirculation within the spray dryer. The nozzle sat flush with the diffuser plate during operation. The suspension was delivered to the nozzle at about 285 g/min at a pressure of about 300 psig. The pump system included a pulsation dampener to minimize pulsation at the nozzle. Drying gas (e.g., nitrogen) was delivered to the diffuser plate at a flow rate of 1850 g/min, and an inlet temperature of 140° C. The evaporated solvent and wet drying gas exited the spray drier at a temperature of 40° C. The coated crystals formed by this process were collected in a cyclone, then post-dried using a Gruenberg single-pass convection tray dryer operating at 40° C. for 4 hours. The properties of the coated crystals after post-drying were as follows:

| Parameter | Value |
|---|---|
| Morphology | Irregular spheres |
| | with evidence of |
| | crystalline particles |
| Crystallinity (% of drug) | 90% ± 10% |
| Mean particle diameter (μm) | 42 |
| *Dv₁₀, Dv₅₀, Dv₉₀ (μm) | 13, 38, 76 |
| Span (D₉₀-D₁₀)/D₅₀ | 1.6 |
| Bulk specific volume (cc/g) | 3.3 |
| Tapped specific volume (cc/g) | 2.2 |
| Hausner ratio | 1.5 |
| Glass Transition Temperature at | 120 (the same as the |
| 5% RH (° C.) | Tg for HPMCAS-HG) |
| Crystallization Temperature (° C.) | None Observed from |
| | 0° C. to 250° C. |

*10 vol % of the particles have a diameter that is smaller than D₁₀; 50 vol % of
the particles have a diameter that is smaller than D₅₀, and 90 vol % of the particles
have a diameter that is smaller than D₉₀.

The ziprasidone coated crystals were evaluated in vitro using a membrane permeation test. An Accurel® PP 1E microporous polypropylene membrane was obtained from Membrana GmbH (Wuppertal, Germany). The membrane was washed in isopropyl alcohol and rinsed in methanol in a sonicating bath for 1 minute at ambient temperature, and then allowed to air dry at ambient temperature. The feed side of the membrane was then plasma-treated to render it hydrophilic by placing a sample of the membrane in a plasma chamber. The atmosphere of the plasma chamber was saturated with water vapor at a pressure of 550 mtorr. A plasma was then generated using radio frequency (RF) power inductively coupled into the chamber via annular electrodes at a power setting of 50 watts for 45 seconds. The contact angle of a drop of water placed on the surface of the plasma-treated membrane was about 40°. The contact angle of a drop of water placed on the permeate side of the same membrane was greater than about 110°.

A permeate reservoir was formed by gluing a sample of the plasma-treated membrane to a glass tube having an inside diameter of about 1 inch (2.54 cm) using an epoxy-based glue (LOCTITE® E-30CL HYSOL® from Henkel Loctite Corp, Rocky Hill, Conn.). The feed-side of the membrane was oriented so that it was on the outside of the permeate reservoir, while the permeate-side of the membrane was oriented so that it was on the inside of the reservoir. The effective membrane area of the membrane on the permeate reservoir was about 4.9 cm². The permeate reservoir was placed into a glass feed reservoir. The feed reservoir was equipped with a magnetic stir bar and the reservoir was placed on a stir plate and the stir rate was set to 100 rpm during the test. The apparatus was placed into a chamber maintained at 37° C. for the duration of the test. Further details of the test apparatus and protocols are presented in co-pending U.S. Patent Application Ser. No. 60/557,897, entitled “Method and Device for Evaluation of Pharmaceutical Compositions,” filed Mar. 30, 2004 (attorney Docket No. PC25968), incorporated herein by reference.

To form the feed solution, a 1.39 mg sample of the coated crystals was weighed into the feed reservoir. To this was added 5 ml of MFD solution previously described, consisting of PBS solution containing 7.3 Mm sodium taurocholic acid and 1.4 mM of 1-palmitoyl-2-oleyl-sn-glycero-3-phosphocholine (0.5% NaTC/POPC). The concentration of ziprasidone in the feed solution would have been 100 μgA/ml, if all of the ziprasidone had dissolved. The feed solution was mixed using a vortex mixer for 1 minute. Before the membrane contacted the feed solution, 5 ml of 60 wt % decanol in decane was placed into the permeate reservoir. Time zero in the test was when the membrane was placed in contact with the feed solution. A 50 ml aliquot of the permeate solution was collected at the times indicated. Samples were then diluted in 250 ml IPA and analyzed using HPLC. The results are shown in Table 3.

As a control, the membrane test was repeated using a 0.5-mg sample of crystalline ziprasidone alone, so that the concentration of drug would have been 100 μgA/ml, if all of the drug had dissolved. These results are also given in Table 3.

TABLE 3

| Formulation | Time (min) | Concentration (μgA/ml) |
|---|---|---|
| Ziprasidone | 0 | 0.0 |
| Coated | 20 | 3.4 |
| Crystals | 40 | 13.2 |
| | 60 | 17.5 |
| | 90 | 25.2 |
| | 120 | 33.3 |
| | 180 | 47.9 |
| | 240 | 48.4 |
| | 360 | 52.4 |
| Crystalline | 0 | 0.5 |
| Ziprasidone | 20 | 5.2 |
| HCl | 40 | 8.1 |
| | 60 | 10.0 |
| | 90 | 11.4 |
| | 120 | 12.9 |
| | 180 | 18.1 |
| | 245 | 20.9 |
| | 360 | 22.6 |

The maximum flux of drug across the membrane (in units of mgA/cm²-min) was determined by performing a least-squares fit to the data in Table 3 from 0 to 60 minutes to obtain the slope, multiplying the slope by the permeate volume (5 ml), and dividing by the membrane area (4.9 cm²). The results of this analysis are summarized in Table 4, and show that the ziprasidone coated crystals provided an initial flux through the membrane that was 2-fold that provided by crystalline ziprasidone alone.

TABLE 4

| | Initial Flux of Ziprasidone |
|---|---|
| Formulation | (mgA/cm²-min) |
| Ziprasidone Coated Crystals | 0.32 |
| Crystalline Ziprasidone HCl | 0.16 |

### Dosage Forms

### Dosage Form DF-1

Dosage Form DF-1 is prepared as follows. First, an enteric coated sustained release core was prepared comprising a matrix sustained-release core containing polymer coated crystals of ziprasidone hydrochloride. The coated crystals were made using the process previously described, and contained 35 wt % of active ziprasidone coated with HPMCAS-HF. The matrix tablets consisted of 30 wt % of the coated crystals, 29 wt % spray-dried lactose, 40 wt % PEO WSRN-10 (100,000 daltons), and 1 wt % magnesium stearate. The tablets were prepared by first blending the coated crystals, lactose, and PEO in a twin-shell blender for 20 minutes, milling using a Fitzpatric M5A mill, and then blending in the twin-shell blender for an additional 20 minutes. The magnesium stearate was then added and the mixture blended again for 5 minutes. The tablets were manufactured using an F press using 381 mg of the mixture using caplet-shaped tooling with dimensions 0.30 inches by 0.60 inches. The tablet cores were compressed to a hardness of about 9.5 kp. The resulting sustained-release matrix tablet contained a total of 40 mg active ziprasidone and had a total mass of about 380 mg.

DF-1 was then coated with an enteric coating. The coating solution consisted of 41.7 wt % Eudragit L30-D55 and 2.5 wt % triethylcitrate in 55.8 wt % water. Coatings were applied in an LDCS-20 pan coater. The coating weight was 10 wt % of the uncoated core weight. The resulting enteric coated sustained-release matrix tablet had a total mass of about 419 mg.

Next, an immediate release coating is applied to the enteric sustained release core. A coating suspension is formed in acetone containing jet-milled ziprasidone and hydroxypropyl methyl cellulose. The drug and polymer collectively are 2 to 15 wt % of the suspension. The suspension is stirred for one hour and is filtered through a 250 μm screen prior to use to remove any particles of polymer that could potentially plug the spray nozzle. The enteric coated sustained release cores are coated in a pan coater. At the conclusion of the spray, the coated dosage forms are dried in a tray drier for one hour at 40° C.

In Vitro Release Tests

An in vitro release test of the sustained release core of DF-1 was performed using direct drug analysis as follows. A sustained release core was first placed into a stirred USP type 2 dissoette flask containing 900 mL of a dissolution medium of a simulated intestinal buffer solution consisting of 50 mM NaH₂PO₄ with 2 wt % sodium lauryl sulfate at pH 7.5 and 37° C. In the flasks, the sustained release core was placed in a wire support to keep the dosage form off of the bottom of the flask, so that all surfaces were exposed to the moving buffer solution and the solutions were stirred using paddles at a rate of 75 rpm. Samples of the dissolution medium are taken at periodic intervals using a VanKel VK8000 autosampling dissoette with automatic receptor solution replacement. The concentration of dissolved drug in the dissolution medium is then determined by HPLC at a UV absorbance of 315 nm using a Zorbax RxC8 Reliance column and a mobile phase consisting of 55% (50 mM potassium dihydrogen phosphate, pH 6.5)/45% acetonitrile. Drug concentration was calculated by comparing UV absorbance of samples to the absorbance of drug standards. The mass of dissolved drug in the dissolution medium was then calculated from the concentration of drug in the medium and the volume of the medium, and expressed as a percentage of the mass of drug originally present in the dosage form. Results are shown in Table 5.

TABLE 5

| | Ziprasidone |
|---|---|
| Time | Released |
| (hrs) | (wt %) |
| 0 | 0 |
| 1 | 17 |
| 2 | 38 |
| 3 | 60 |
| 4 | 79 |
| 5 | 95 |
| 6 | 100 |
| 8 | 100 |
| 10 | 100 |

The results show that the sustained release core released 90 wt % of the drug during the first five hours after administration to the in vitro test media.

The terms and expressions which have been employed in the foregoing specification are used therein as terms of description and not of limitation, an there is no intention, in the use of such terms and expressions, of excluding equivalents of the features shown and described or portions thereof, it being recognized that the scope of the invention is defined and limited only by the claims which follow.

1. A dosage form comprising:
(a) an immediate release portion comprising a low-solubility drug, said low-solubility drug having a dose to aqueous solubility ratio of at least about 100 ml;
(b) a sustained release core comprising said low-solubility drug, said sustained release core being surrounded by an enteric coating;
wherein said sustained release core is sufficiently large to be retained in the stomach for a period of time, said sustained release core releases at least 90 wt % of said drug in said core over a release period of from about 1 hour to about 8 hours, and said drug in said sustained release core is in a solubility-improved form.

2. A dosage form comprising:
(a) an immediate release portion comprising a low-solubility drug, said low-solubility drug having a dose to aqueous solubility ratio of at least about 10 ml,
(b) a sustained release core comprising said low-solubility drug, said sustained release core being surrounded by an enteric coating;
wherein said sustained release core is sufficiently large to be retained in the stomach for a period of time, said sustained release core releases at least 90 wt % of said drug in said core over a release period of from about 1 hour to about 8 hours, and said drug has a clearance half life of less than about 12 hours.

3. The dosage form of claim 2 wherein said drug has a dose-to-solubility ratio of greater than about 100 ml.

4. The dosage form of claim 1 wherein said drug has a dose-to-aqueous solubility ratio of greater than about 1000 ml.

5. The dosage form of claim 1 wherein said drug has a clearance half life of less than about 12 hours.

6. The dosage form of claim 2 wherein said drug has a clearance half life of less than 8 hours.

7. The dosage form of claim 1 wherein said drug is in the form of particles having a mean particle size of less than about 100 microns.

8. The dosage form of claim 1 further comprising a precipitation inhibiting polymer.

9. The dosage form of claim 1 wherein said sustained release core and said enteric coating have a mass of at least about 400 mg.

10. The dosage form of claim 1 wherein said dosage form has a longest dimension of at least about 5 mm.

11. The dosage form of claim 1 wherein said sustained release core has a release period of from about 1 to about 6 hours.

12. The dosage form of claim 1 wherein said sustained release core is selected from the group consisting of a matrix sustained release core, an osmotic sustained release core, and a capsule.

13. The dosage form of claim 1 wherein said immediate release portion is selected from the group consisting of a layer, a coating, a powder, multiparticulates and granules.

14. The dosage form of claim 13 wherein said immediate release portion comprises a coating surrounding said enteric coating.

15. A dosage form according to claim 1, wherein the low-solubility drug is ziprasidone or a pharmaceutically acceptable salt thereof.

16. The dosage form of claim 2 wherein said drug has a dose-to-aqueous solubility ratio of greater than about 1000 ml.

17. The dosage form of claim 5 wherein said drug has a clearance half life of less than 8 hours.

18. The dosage form of claim 6 wherein said drug is in the form of particles having a mean particle size of less than about 100 microns.

19. The dosage form of claim 6 further comprising a precipitation-inhibiting polymer.

20. The dosage form of claim 2 wherein said sustained release core and said enteric coating have a mass of at least about 400 mg.

21. The dosage form of claim 2 wherein said dosage form has a longest dimension of at least about 5 mm.

22. The dosage form of claim 2 wherein said sustained release core has a release period of from about 1 to about 6 hours.

23. The dosage form of claim 2 wherein said sustained release core is selected from the group consisting of a matrix sustained release core, an osmotic sustained release core, and a capsule.

24. The dosage form of claim 2 wherein said immediate release portion is selected from the group consisting of a layer, a coating, a powder, multiparticulates and granules.

25. A dosage form according to claim 2, wherein the low-solubility drug is ziprasidone or a pharmaceutically acceptable salt thereof.

26. A dosage form according to claim 3, wherein the low-solubility drug is ziprasidone or a pharmaceutically acceptable salt thereof.

27. A dosage form according to claim 4, wherein the low-solubility drug is ziprasidone or a pharmaceutically acceptable salt thereof.

28. A dosage form according to claim 5, wherein the low-solubility drug is ziprasidone or a pharmaceutically acceptable salt thereof.

29. A dosage form according to claim 6, wherein the low-solubility drug is ziprasidone or a pharmaceutically acceptable salt thereof.

30. A dosage form according to claim 7, wherein the low-solubility drug is ziprasidone or a pharmaceutically acceptable salt thereof.

31. A dosage form according to claim 8, wherein the low-solubility drug is ziprasidone or a pharmaceutically acceptable salt thereof.

32. A dosage form according to claim 9, wherein the low-solubility drug is ziprasidone or a pharmaceutically acceptable salt thereof.

33. A dosage form according to claim 10, wherein the low-solubility drug is ziprasidone or a pharmaceutically acceptable salt thereof.

34. A dosage form according to claim 11, wherein the low-solubility drug is ziprasidone or a pharmaceutically acceptable salt thereof.

35. A dosage form according to claim 12, wherein the low-solubility drug is ziprasidone or a pharmaceutically acceptable salt thereof.

36. A dosage form according to claim 13, wherein the low-solubility drug is ziprasidone or a pharmaceutically acceptable salt thereof.

37. A dosage form according to claim 14, wherein the low-solubility drug is ziprasidone or a pharmaceutically acceptable salt thereof.

38. A dosage form according to claim 16, wherein the low-solubility drug is ziprasidone or a pharmaceutically acceptable salt thereof.

39. A dosage form according to claim 17, wherein the low-solubility drug is ziprasidone or a pharmaceutically acceptable salt thereof.

40. A dosage form according to claim 18, wherein the low-solubility drug is ziprasidone or a pharmaceutically acceptable salt thereof.

41. A dosage form according to claim 19, wherein the low-solubility drug is ziprasidone or a pharmaceutically acceptable salt thereof.

42. A dosage form according to claim 20, wherein the low-solubility drug is ziprasidone or a pharmaceutically acceptable salt thereof.

43. A dosage form according to claim 21, wherein the low-solubility drug is ziprasidone or a pharmaceutically acceptable salt thereof.

44. A dosage form according to claim 22, wherein the low-solubility drug is ziprasidone or a pharmaceutically acceptable salt thereof.

45. A dosage form according to claim 23, wherein the low-solubility drug is ziprasidone or a pharmaceutically acceptable salt thereof.

46. A dosage form according to claim 24, wherein the low-solubility drug is ziprasidone or a pharmaceutically acceptable salt thereof.
